(19) United States
(12) Patent Application Publication
(10) Pub. No.: US 2022/0370328 A1
(43) Pub. Date: Nov. 24, 2022

(54) Clear Nail Top Coat Compositions And Methods Of Making

(71) Applicant: ILNP Cosmetics Inc., Las Vegas, NV (US)

(72) Inventor: Samuel A. Davis, Las Vegas, NV (US)

(73) Assignee: ILNP Cosmetics Inc., Las Vegas, NV (US)

(21) Appl. No.: 17/769,678

(22) PCT Filed: Aug. 27, 2020
(86) PCT No.: PCT/US2020/048246

§ 371 Date: Apr. 15, 2022

Related U.S. Application Data
(60) Provisional application No. 62/916,676, filed on Oct. 17, 2019.

(51) Int. Cl. A61K 8/73; A61K 8/81; A61K 8/34; A61Q 3/02

(52) U.S. Cl. CPC A61K 8/731; A61K 8/8135; A61K 8/34; A61Q 3/02

(57) ABSTRACT

Clear nail polish top coat formulations prepared without the use of toxic compounds, such as toluene, are provided. The compositions utilize cellulose acetate butyrate (CAB) as a main resin a discussed are the effects of numerous solvents, plasticizers, co-resins, and/or additives on CAB's solubility. For example, the type and ratio of solvents used are related to the selected CAB grade's molar ratio of butyryl, acetyl, and hydroxyl R-groups. Performances measures include, but are not limited to, transmitted haze, reflected haze, hardness, viscosity, shrinkage, tensile strength, and elastic modulus. Additionally, the effect of filtration on the transmitted haze is also discussed.

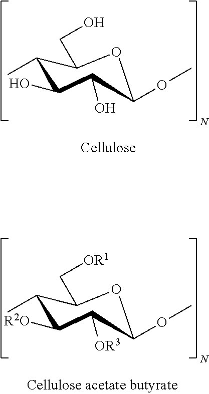

### FIELD OF TECHNOLOGY

This disclosure relates generally to compositions and methods of making clear nail top coat, and, more particularly, to compositions and methods of making clear nail top coats with cellulose acetate butyrate (CAB or acetobutyrate cellulose) and which do not contain toluene.

### BACKGROUND OF INVENTION

A clear nail top coat is used to cover a nail polish color coat with a clear, glossy, and durable coating. CAB was first utilized in top coat nail lacquers by Martin et al. (U.S. Pat. No. 5,130,125; hereinafter '125) in July 1992. '125's composition requires about 31% toluene in the nail top coat in order to create clear solutions. Seche Vite™ is a clear nail top coat formulated based on '125's composition and is renowned for its liquid clarity, fast dry time and glossy finish. However, toluene is classified to have reproductive/teratogenic toxicity. Toluene is suspected of damaging fertility or the unborn child (toluene SDS). Toluene has the possibility of creating explosive reactions when mixed with common household items such as silver and acetic acid, a component of vinegar (toluene SDS). Toluene also has numerous material compatibility requirements that limit its use (such as requiring a glass or Teflon™ container) and its non-conductive nature can result in static buildup which can cause it to ignite at room temperature.

Despite toluene's toxicity, flammability, and unpleasant glue odor, Seche Vite™ has appeal due to the clarity of the solution. Although competing products now exist which eliminate toluene, the same clarity as Seche Vite™ in the final solution has yet to be achieved. Thus, competitor products are shrouded in frosted bottles or colorants are added to hide the cloudiness. This problem is summarized best in Weisman (U.S. Pat. No. 5,747,019; hereinafter '019), in which CAB is replaced with cellulose acetate propionate (CAP), and which recites: “if toluene is exchanged for ethyl acetate the resulting composition is slightly cloudy . . . To mitigate this ‘cloudy’ problem one must slightly pigment the composition or sell the composition in a frosted bottle.” (See column 2, lines 23-30 and 32-44).

There are a significant number of competitors in the nail polish top-coat space. Examples of contemporary top coats that perform well are as follows:

LVX Gel Top Coat uses the following ingredients:
a) Butyl Acetate
b) Ethyl Acetate
c) Acrylates Copolymer
d) Nitrocellulose (NC)
e) Acetyl Tributyl Citrate
f) Adipic Acid/Neopentyl Glycol/Trimellitic Anhydride Copolymer
g) Etocrylene
h) Isopropyl Alcohol (IPA)
i) Trimethylpentanediyl Dibenzoate
j) Violet 2

LVX Gel Top Coat contains a colorant and does not utilize CAB as a resin (film former). Although the use of CAB in top coats is difficult due to lack of clarity, CAB is still a preferred main resin because it has high UV resistance, low flammability, bonds well to underlying coatings, and can be incorporated at high percentages to provide thick coatings with good viscosity, spreading, and leveling characteristics. CAB also elongates without breaking more easily than nitrocellulose, which helps to form a stable film when underlying layers are still drying and shrinking. CAB is also compatible with many different resins, plasticizers and solvents and can improve dry-to-touch time and coating adhesion. This formula also uses acrylates copolymer as the main film former, which, depending on the formulation, is generally softer than CAB, and reduces scratch resistance of the nail top coat. As above, many commercial top coat formulas also contain Nitrocellulose (or Cellulose Nitrate) resin. The dis-advantages of using Nitrocellulose as a resin are a high potential for yellowing and increased manufacturing safety risk due to the classification of Nitrocellulose as an explosive.

Another product is Seche Vite™ Dry Fast Top Coat mentioned above, the ingredients of which are:
a) Butyl Acetate
b) Toluene
c) CAB
d) IPA
e) Trimethyl Pentanyl Diisobutyrate
f) Benzophenone-1

The main disadvantage of this formula is the presence of a large percentage of toxic toluene. This formula also has a high shrinkage rate and is reputed to shrink inwards from the edges of the nail plate, sometimes dragging the underlying layers. A slower drying rate than other formulas was also observed, due to the low volatility of the main solvent n-butyl acetate and medium volatility of the co-solvent toluene.

Another product is INM Out The Door Top Coat which uses the following ingredients:
a) Ethyl Acetate
b) SD Alcohol 40B (Alcohol denat.)
c) Butyl Acetate
d) CAB
e) Acrylates Copolymer
f) Trimethyl Pentanyl Diisobutyrate
g) Sucrose Benzoate
h) Triphenyl Phosphate (TPP)
i) NC
j) IPA
k) Benzophenone-1
l) Dimethyl Polysiloxane
m) Violet 2 (CI 60725)
n) Blue 1 (CI 42090)

This product utilizes multiple colorants and the contents are completely covered by a label. The liquid is cloudy and has a noticeable bluish hue.

Another example is Essie Good to Go Top Coat which uses the following ingredients:
a) Butyl Acetate
b) Ethyl Acetate
c) CAB
d) IPA
e) Trimethyl Pentanyl Diisobutyrate
f) Adipic Acid/Neopentyl Glycol/Trimellitic Anhydride Copolymer
g) CI 60725 (Violet 2)
h) Benzophenone-1
i) Dimethicone
j) D44603/5

This is another example in which multiple colorants and bottle design mask the cloudiness imparted by CAB when toluene is replaced with ethyl and butyl acetate as primary solvents.

Thus, a nail top coat composition incorporating CAB as a main resin is needed which does not include highly toxic compounds such as toluene. It must also be completely clear, haze-free and colorless, despite the use of organic solvents and/or plasticizer additives, and achieved without adding colorants or using frosted bottles. The composition must also be fast-drying; self-leveling; hard; flexible; strong; high gloss; scratch-resistant; UV resistant; stable over time; bond well over an underlying lacquer layer; thick; smooth; have a pleasant odor; shrink resistant; and resistant to common household chemicals (such as grease, vinegar, water, soap and salt). Lastly, the top coat should be cruelty-free and vegan and filtered of any impurities before packaging into nail polish bottles.

### SUMMARY OF INVENTION

Several novel formulas are described which create clear, fast-drying, non-toxic (non-toluene and non-phthalates, and, in the most preferred embodiments, non-TPP) nail top coat formulas with good hardness, strength, flexibility, bonding, smoothness, wear and impact resistance, spreadability, odor, viscosity and gloss; and low shrinkage and reflected haze. The novel formulas are classified as listed below by the ingredients they contain and whether filtering is needed to reduce the Haze(C/2) to 2.1% or below:

Formulas that contains CAB as the main resin, and contain acrylates copolymer or other co-resins, such as nitrocellulose or tosylamide/epoxy resin and contain a plasticizer(s) and other additive(s). These formulas require the use of one or more volatile butyrate ester solvents (such as EB and MB) but does not require filtering to achieve the desired clarity. An example which does not require filtering is provided in Table 20, Formula SR-28-F.

Formulas that contain CAB as the main resin, but do not contain a co-resin and contain a plasticizer(s) and other additive(s). These formulas require both the use of one or more volatile butyrate ester solvents (such as EB and MB) and filtering to achieve the desired clarity and various other characteristics. Examples of these formulas are provided in Tables 7 (formulas A1 to A3), Table 9 (formulas TC-5-F through TC-7-F), and Table 21.

Formulas that contain CAB as the main resin and contain a co-resin(s) and contain one or more volatile butyrate ester solvents (such as EB and MB) and may or may not contain a plasticizer(s) and other additives and does require filtering. Examples of these formulas are provided in Table 8 (top coats B2 and C2), Table 9 (top coats TC-1-F through TC-3-F), Table 11, Table 13 (formulas CR2-2-F, CR2-3-F, CR3-2-F, and CR3-3-F), Table 15 (PR-1-F), Table 16, Table 17, Table 20 (formulas SR-26-F, SR-27-F, SR-29-F, SR-30-F) and Table 22 (formulas TCSV66-1-12-F and TCSV66-2-8-F).

Formulas that contain CAB as the main resin and contain a co-resin(s) and do not contain one or more volatile butyrate ester solvents (such as EB and MB) and contain a plasticizer(s) and other additives and require filtering. Examples of these formulas are provided in Table 10, Formulas SOL-1 and TC-4-F.

CAB-381-0.5 is preferred because it has the least haze(See CHF-1/2/3 and CHB-1/2/3 in Table 18).

All the solvents affect the odor of the composition. In particular, MB, EB, MP, iBA and SDA40B all have a significant impact on odor. However, a small amount of fragrance can also be added around 0.5% without causing negative effects on mechanical properties while improving odor.

SB was found to be a good additive for improving clarity, increasing viscosity, and increasing gloss and reducing reflective haze. It can be added up to about 7-8% before mechanical or other properties of the film start to suffer.

The addition of the smoothers dimethicone (Xiameter PMX-200 10 Cst) or Dowsil 556 (Phenyl Trimethicone) results in improved gloss, reflective haze, impact/flex resistance, and a smoother dry surface without compromising hardness, clarity or drying rate.

### BRIEF DESCRIPTION OF THE DRAWINGS

The embodiments of this invention are illustrated by way of example and not limitation in the figures of the accompanying drawings, in which like references indicate similar elements and in which:

FIG. 1 shows the molecular structure of cellulose and CAB.

FIG. 2 shows CAB substitution data.

FIG. 3 shows filtration results for particular samples of clear top coat.

FIG. 4 is a graph showing filtered (0.22 μm) versus unfiltered % Haze(C/2) for CAB resin top coats with and without Acrylates Copolymer co-resin. The graph shows an average of 48% reduction in % Haze(C/2).

FIG. 5 is a graph showing filtered (0.22 μm) versus unfiltered % Haze(C/2) for CAB resin top coats with Acrylates Copolymer co-resin. The graph shows an average of 52% reduction in % Haze(C/2).

FIG. 6 is a graph showing filtered (0.22 μm) versus unfiltered % Haze(C/2) for CAB resin top coats without Acrylates Copolymer co-resin. The graph shows an average of 46% reduction in % Haze(C/2).

FIG. 7 is a diagram showing an exemplary transmitted % Haze(C/2) measurement device in accordance with ASTM D1003-13.

FIG. 8 is a graph showing transmitted % Haze(C/2) versus Wt % CAB-381-2 in n-Butyl Acetate. Similar results were obtained for ethyl acetate.

FIG. 9 is a graph showing % Haze(C/2) versus Wt % CAB-381-2 in n-Butyl Acetate and sucrose benzoate. The clarity of CAB-381-2 in nBA is improved by incorporating sucrose benzoate into the solvent. The haze is nearly half of what it is with only nBA for a given Wt % CAB-381-2.

FIG. 10 is a flow diagram showing exemplary weighing 1000 and mixing 1010 processes in addition to optional filtration 1020 and bottling 1030 processes.

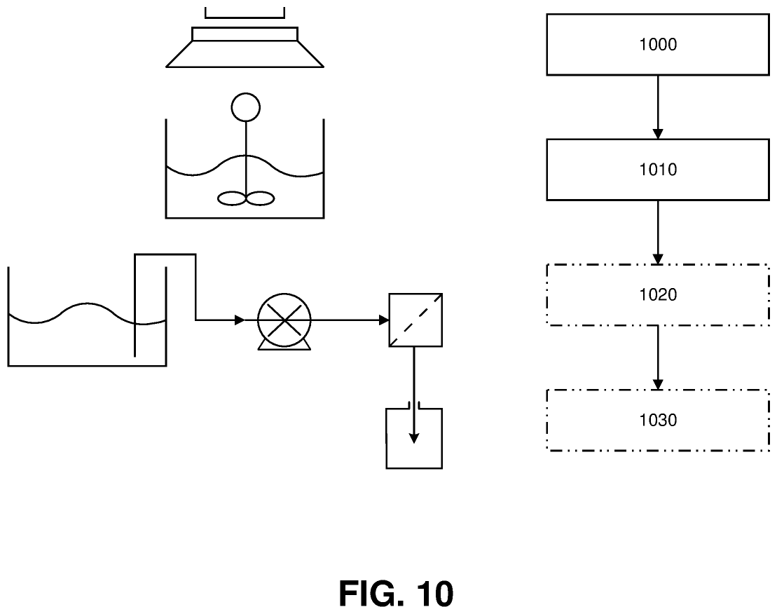

FIG. 11 is a pencil hardness scale and a diagram showing an exemplary mechanical holder with sharpened drawing lead used for testing hardness in accordance with ASTMD3363. A 750 g force was used to test hardness with this method.

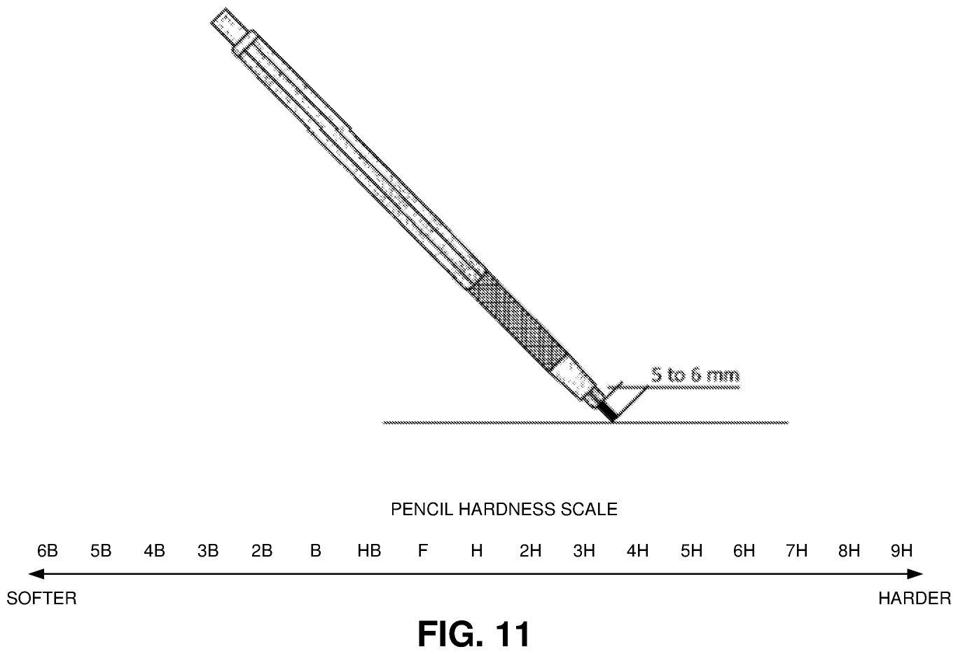

FIG. 12 is a diagram showing the mechanism of a Brinell Hardness test in accordance with ASTM E 10. Force (F) was equivalent to 500 g and Diameter (D) was equivalent to 12.7 mm during testing.

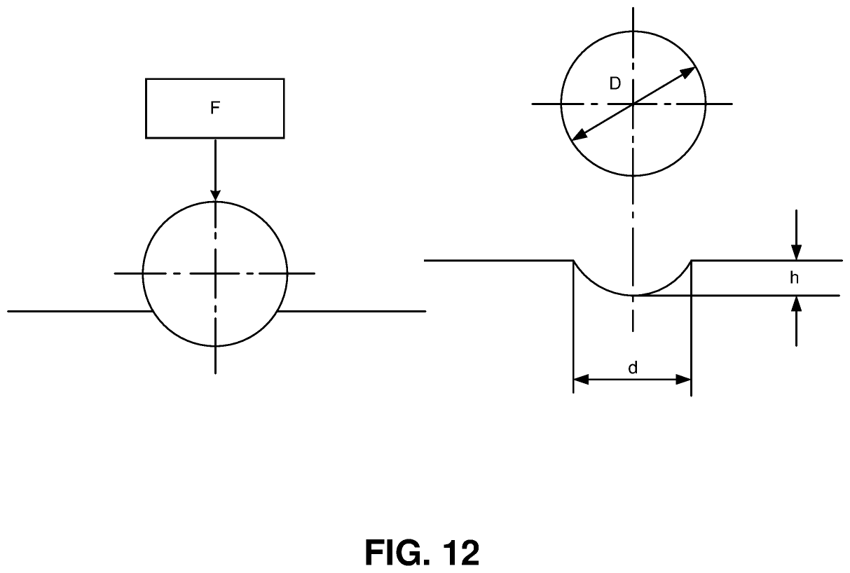

FIG. 13 is a graph showing % Haze(C/2) transmitted in CAB-381-0.5 solutions versus proportion of methyl butyrates to total butyrate content.

FIG. 14 is a graph showing a reducing effect of higher alcohol content on transmitted haze.

FIG. 15 is a graph showing an optimum alcohol content calculation.

FIG. 16 is a graph showing that methyl butyrate lowers viscosity and ethyl butyrate raises viscosity.

FIG. 17 is a graph showing increased % Haze(C/2) and viscosity with increased Wt % of CAB.

FIG. 18 is a graph showing % Haze(C/2) versus calculated Relative Energy Difference (RED) for unfiltered top coat with and without acrylates copolymer.

FIG. 19 is a graph showing measured versus calculated % Haze for all formulas.

FIG. 20 is a graph showing measured versus calculated % Haze for all formulas with acrylates copolymer.

FIG. 21 is a graph showing polarity versus hydrogen bonding Hansen Solubility Parameter (HSP) for an exemplary formula SR-28-F.

FIG. 22 is a graph showing dispersion versus hydrogen bonding HSP for an exemplary formula SR-28-F.

Other features of the present embodiments will be apparent from the accompanying drawings and from the detailed description that follows.

### DETAILED DESCRIPTION

Numerous formulations of a fast-drying nail polish top coat are provided which do not use the highly toxic toluene. Compositions involving CAB have been provided which, inter alia, factor in the solubility of CAB when used with various solvents and/or plasticizers. Mixtures are provided which utilize solvents in proportion to the molar ratio of butyryl, acetyl, and hydroxyl groups bound to monomer units of different grades of CAB, which is provided by Eastman Chemical Company. Additionally, compositions utilizing CAB as a main resin and incorporating various solvents, plasticizers, and/or additives without toluene are disclosed which maintain clarity within defined CAB weight percentages, based on the grade of CAB.

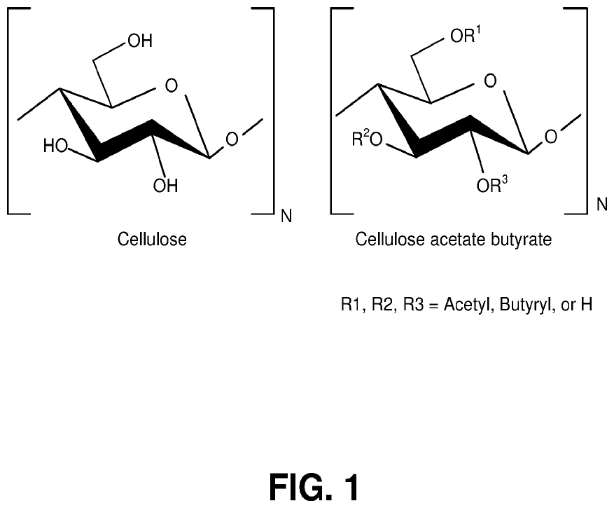

CAB is a biological polymer derived from the cellulose found in plants. Cellulose is a polysaccharide formed via a network of β-linked D-glucose monomers. Each glucose monomer contains three hydroxyl groups. The CAB structure is produced by substituting each (of 3) hydroxyl groups of D-glucose with acetyl, butyryl, and hydrogen substituents (R-groups). The substitution of R-groups on the CAB structure occurs at the positions shown in FIG. 1. The CAB molecular structure can be modified to contain varying ratios of R-groups having a direct effect on CAB's solubility and viscosity in a given solvent. The flexibility, solubility and compatibility of CAB improves with increasing butyryl substitution. One approach to finding ideal solvent/plasticizer mixtures capable of dissolving CAB, is matching to some extent the molar percentage of each of CAB's R-group substituents to that of the solvent/plasticizer. In this way of thinking, the butyryl R group is most similar to a butyrate ester such as methyl or ethyl butyrate (synonymous with methyl or ethyl butanoate). Other branched and straight chain butyrate esters with higher molecular weight could be selected for this purpose, such as, but not limited to, isobutyl isobutyrate, n-butyl butyrate, cyclohexyl butyrate, isopentyl butyrate, benzyl butyrate, 1-naphthyl butyrate, octyl butyrate, propyl butyrate, n-Amyl butyrate, hexyl butyrate, heptyl butyrate, decyl butyrate, cis-3-Hexenyl butyrate, isobutyl butyrate, trimethyl pentanyl diisobutyrate, sucrose acetate isobutyrate, and so on. However, as the molecular weight of the butyrate ester increases, it's volatility will generally decrease, which is not usually advantageous for a fast-drying top coat such as being disclosed herein. Further, the acetyl R group is most similar to an acetate ester, such as methyl acetate, ethyl acetate, propyl acetate and butyl acetate. Also, the H—R group creates an —OH group on the cellulose backbone and is most similar to an alcohol, such as ethanol, propanol and butanol. Lastly, the 6-ringed structure of the cellulose backbone of CAB to which the R groups are attached indicates some compatibility with a high dispersion ringed structure, such as a phenyl group. This indicates strong compatibility with the plasticizers triphenyl phosphate (TPP), a phosphate ester, which contains 3 phenyl groups and Trimethylpentanediyl dibenzoate with 2 phenyl groups. The high percentage of butyryl substitution in CAB also indicates good compatibility with the plasticizer Trimethyl pentanyl diisobutyrate and Sucrose Acetate Isobutyrate (SAIB). Although not a plasticizer, Sucrose benzoate also has a high number of phenyl groups which indicates good solvency for CAB.

Other compounds with a proportional number of phenyl groups may be used without compromising (to improve) the solubility of CAB. Such compounds may include, but not be limited to:
a) Film forming INCI (International Nomenclature of Cosmetic Ingredients) dictionary ingredients, such as isopropylidenediphenyl bisoxyhydroxypropyl methacrylate, tosylamide/epoxy resin, tosylamide/formaldehyde resin, styrene/acrylates copolymer, and isopropylidenediphenyl bisoxyhydroxypropyl methacrylate/TMDI copolymer.
b) Perfuming INCI ingredients, such as 1,1-dimethyl-2-phenylethyl isobutyrate, 1,1-dimethyl-3-phenylpropyl isobutyrate, 1,3-dimethyl-3-phenylbutyl acetate, 1-phenylpropyl acetate, benzyl phenylacetate, butyl phenylacetate, ethyl phenylacetate, isobutyl phenylacetate, isopropyl phenylacetate, propyl phenylacetate, and diphenyl ether.
c) INCI ingredients for gloss, shine and smoothing, such as phenyl trimethicone, phenyl dimethicone, and diphenyl dimethicone.
d) INCI ingredients for UV curing, such as ethyl trimethylbenzoyl phenylphosphinate, hydroxycyclohexyl phenyl ketone, and trimethylbenzoyl diphenylphosphine oxide.
e) INCI ingredients for UV adsorption and UV stabilizers, such as BHT (butylated hydroxy toluene), benzophenone-1, etocrylene, hydroquinone, and p-hydroxyanisole.
f) Other ingredients, such as xylene, ethylbenzene, acetophenone, benzyl benzoate, phenylpropane, phenyl acetone, phenylpropanol, tetraphenyl silane, triphenylsilane, triphenylsilanol, 2,2,2-triphenylacetophenone, (carbethoxymethylene)triphenylphosphorane, (triphenylphosphoranylidene)acetaldehyde, methyl triphenylmethyl ether, 1,3,5-triphenylbenzene, triphenylmethanol, triphenylamine, triphenylborane, triphenyl phosphite, triphenyl phosphine oxide, polystyrene, Poly(styrene-co-methyl methacrylate), phenyl methacrylate, benzyl methacrylate, fluorescein O-methacrylate, poly(benzyl methacrylate), ethylene glycol phenyl ether methacrylate, poly(ethylene glycol) 2,4,6-tris(1-phenylethyl)phenyl ether methacrylate, methyl (triphenylphosphoranylidene)acetate, sodium/potassium tetraphenylborate, triphenylene, triphenylsulfonium acetate, triphenylsulfonium chloride solution, tetraphenylphosphonium chloride/bromide/etc., phenyl sulfate, diphenyl sulfate (diphenyl sulphone), tetraphenyl ethane, tetraphenyl methane, tetraphenyl germane, triphenyl germanium chloride, 1,1,2,2-tetraphenyl-1,2-ethanediol, tetraphenylcyclopentadienone/1,2,3,4-tetraphenyl-1,3-cyclopentadiene, tetraphenyltin, tetraphenylbiphosphine, ammonium tetraphenylborate, zinc TPP, (4S,5S)-2,2-dimethyl-α,α,α′,α′-tetraphenyldioxolane-4,5-dimethanol, 1,2,3,4-tetraphenylnaphthalene, meso-tetraphenylporphyrin, 5,10,15,20-tetraphenyl-21h,23h-porphine cobalt(ii), 5,10,15,20-tetraphenyl-21h,23h-porphine iron(iii) chloride, 1,1,2,2-tetraphenylethylene, 1,1,4,4-tetraphenyl-1,3-butadiene, 1,2,3,4,5-pentaphenyl-1,3-cyclopentadiene, 1,1,1,2,2-pentaphenyl-2-(p-tolyl)disilane, 1,1,1,2,2-pentaphenyldisilane, 1,2,3,4,5-pentaphenyl-pentane-1,5-dione, pentaphenylbenzene, ethyl 3-oxo-4-(triphenylphosphoranylidene)butyrate, triphenylaluminum solution, and (r)-(+)-1,1,2-triphenyl-1,2-ethanediol 2-acetate.

The non-INCI ingredients above have not been vetted for potential safety issues, cost, availability and so on. The compounds above may serve more than just the one purpose of improving CAB solubility in the formula. For instance, organometallic compounds such as tetraphenyl germane may serve to improve solubility of CAB and hardness, smoothness and gloss of the dry film due to the metallic component.

A range of CAB grades are defined by varying molar ratios of the R-groups on the CAB structure thus producing a range of solubility and viscosity in solvent/plasticizer mixtures. Eastman produces thirteen CAB grades which have varying butyryl, acetyl and hydroxyl R-group substitution. Three distinct CAB grades, CAB-381-0.5, CAB-551-0.2, and CAB-381-2 were selected to test in a spectrum of solvent mixtures, after eliminating the other grades based on solubility, viscosity or mechanical properties. The first two digits of the CAB product code are roughly the butyryl wt %. The third digit is roughly the OH wt %. The last digits after the last dash represent the viscosity in seconds per the ASTM falling ball viscosity test using solution A (which is 20 Wt % CAB, 72 Wt % Acetone and 8 Wt % Ethanol). Different CAB grades may be used to change the viscosity and adhesion of the top coat or mechanical properties of the film. For instance, CAB-381-2 has a high glass transition temperature and a higher viscosity than CAB-381-0.5 or CAB-551-0.2. CAB-551-0.2 has the lowest viscosity of these three grades and a higher butyryl content, which usually makes it easier to dissolve and it behaves somewhat like a plasticizer, improving the spreading, adhesion, and leveling of the film. CAB grades are further described by their molar ratio of butyryl, acetyl, and hydroxyl R-groups. In a preferred embodiment, an ideal film was created using a clear top coat composition comprising (on a CAB weight basis) 76.06% CAB-381-0.5, 14.79% CAB-551-0.2, and 9.15% CAB-381-2. A large proportion of this CAB content contains a high percentage of butyryl R-groups, an issue ignored by the prior art.

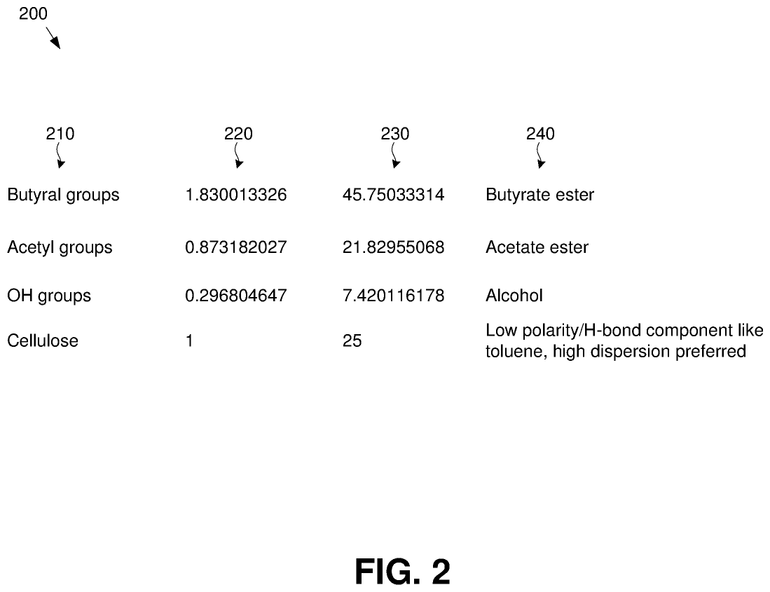

Referring to FIG. 2, a table showing R-group properties for the above-described CAB mixture is shown and is used to determine an ideal solvent and plasticizer mixture. Column 210 corresponds to the breakdown (of an average CAB monomer into its constituent 3 R-groups and the cellulose backbone) of R-groups per monomer, column 220 shows how many mols of the R-group per monomer are exhibited in the CAB mixture on average. As such, the sum of column 220 adds up to 4, representing the 3 mols of R groups and 1 mol of cellulose backbone per CAB monomer. Column 230 calculates the mole percentage of the R-groups and cellulose backbone (i.e., the mols of the constituent divided by 4 and multiplied by 100), and column 240 suggests corresponding solvents which may be utilized to solubilize CAB, based on the principal of like dissolves like. Because the ratio of substituent groups affects the relative solubility of each CAB grade in a solvent mixture, the ideal solvent mixture to produce the best clarity must contain one or more volatile solvents and plasticizers which are in proportion to the R groups (butyral, acetyl, and hydroxyl substituents) and cellulose backbone, when one or more CAB grades are added in solution.

In cosmetics, solubility of solids or resins in liquid/solvent mixtures is often predicted using Hansen Solubility Parameters (HSP's). HSP theory states that each chemical is described by three solubility parameters for dispersion (δ_{d}), polarity (δₚ) and hydrogen bonding (δ_{H}). The square of each parameter combines to define the total solubility parameter (δ, often referred to as the Hildebrand solubility parameter).

δ²=δ_{d}²+δₚ²+δ_{H}² Equation 1:

Further, the resin or solid has a sphere of solubility with a radius of R₀. Solvent mixtures that fall within the resin's sphere of solubility will generally be good solvents for the resin. The closer the liquid mixture to the center of the sphere, the better solvent it is for the resin/solid. The solubility parameters and radius of solubility are published for many solvents, plasticizers, and resins. When the resins are being dissolved in a mixture/blend, the volume-averaged values are used for the solubility parameters (where, P is the blend solubility parameter (δ_{d}, δₚ or δ_{H}), and Pᵢ is the solubility parameter and vᵢ is the volume fraction of a molecule and N is the number of molecules in the blend).

$$P = {\sum }_{i = 1}^{N} {v}_{i} {P}_{i} \qquad Equation 2$$

Once the solubility parameters are calculated for the liquid/blend, then the distance (Rₐ) between the resin and blend are calculated. This distance represents how close the blend is to the center of the sphere of solubility for the resin. The smaller the distance, the more compatible the resin and blend, and the more likely that the resin will be completely dissolved. The Relative Energy Difference (RED) is used to further define good and bad blends for the resin. If RED is <1 it is inside the resins sphere of solubility and is predicted to dissolve the resin. If RED is >1, then the blend is not predicted to dissolve the resin. The smaller the value of RED the more the solvents power for dissolving the resin.

$${R}_{a} = \sqrt{4 {( {δ}_{d , 1} - {δ}_{d , 2} )}^{2} + {( {δ}_{p , 1} - {δ}_{p , 2} )}^{2} + {( {δ}_{H , 1} - {δ}_{H , 2} )}^{2}} \qquad Equation 3$$

RED=Rₐ/R₀ Equation 4:

The table below lists HSP values for various solvents, resins and plasticizers (MPa^{1/2})

TABLE 2
HSP's for various compounds (MPa\hat{ }½).

| Compound | Class | δ_{d} | δₚ | δₕ | R₀ |
|---|---|---|---|---|---|
| CAB | Resin | 16.6 | 12 | 6.7 | 10.2 |
| Nitrocellulose RS ½ sec | Co-Resin | 15.41 | 14.73 | 8.84 | 11.46 |
| Acrylates Copolymer | Co-Resin | 16.0 | 7.7 | 5.8 | 8.59 |
| n-Butyl Acetate | Solvent | 15.8 | 3.7 | 6.3 | |
| Ethyl Acetate | Solvent | 15.8 | 5.3 | 7.2 | |
| n-Propyl Acetate | Solvent | 15.3 | 4.3 | 7.6 | |
| Iso-Butyl Acetate | Solvent | 15.1 | 3.7 | 6.3 | |
| Methyl Butyrate | Solvent | 15.5 | 5.6 | 5 | |
| Ethyl Butyrate | Solvent | 15.5 | 6.6 | 5 | |
| Toluene | Solvent | 18 | 1.4 | 2 | |
| Iso-Propanol | Solvent | 15.8 | 6.1 | 16.4 | |
| Ethanol | Solvent | 15.8 | 8.8 | 19.4 | |
| Methyl Propionate | Solvent | 15.5 | 6.5 | 7.7 | |
| Methyl Acetate | Solvent | 15.5 | 7.2 | 7.6 | |
| Triphenyl Phosphate | Plasticizer | 20.1 | 6.4 | 6.8 | |
| Trimethylpentanediyl | Plasticizer | 17.98 | 5.69 | 8.09 | |
| dibenzoate | | | | | |
| Trimethyl pentanyl | Plasticizer | 16.84 | 4.41 | 9.06 | |
| diisobutyrate | | | | | |
| Propylene Carbonate | Plasticizer | 20 | 18 | 4.1 | |
| Acetyl Tributyl Citrate | Plasticizer | 16.8 | 3.4 | 4.5 | |
| Sucrose Benzoate | Other | 19.4 | 10.5 | 12.9 | |

Another way to use HSP data in Table 2 above, is to calculate the RED of the plasticizer with the resin using Equation 4 above. According to this calculation result (see table 3 below), Trimethylpentanediyl Dibenzoate is the most compatible plasticizer for CAB, PMMA, and NC resins, because the RED is the lowest for all three resins.

TABLE 3
Compatibility of Plasticizer with Resins.

| | RED of Plasticizer with Resin | | |
|---|---|---|---|
| Plasticizer | CAB | NC | PMMA |
| Triphenyl Phosphate | 0.879 | 1.109 | 0.594 |
| Trimethylpentanediyl dibenzoate | 0.688 | 0.909 | 0.587 |
| Propylene Carbonate | 0.925 | 0.946 | 1.008 |
| Trimethyl pentanyl diisobutyrate | 0.781 | 0.935 | 0.846 |
| Acetyl Tributyl Citrate | 0.871 | 1.086 | 0.997 |

The effect of temperature on solubility varies. Most solids/resins become more soluble with increasing temperature and others have decreasing solubility with increasing temperature (such as calcium/magnesium salts of carbonate and sulfate). Increasing temperature to dissolve a resin may not be desirable due to the flammable nature of volatile solvents, impact on air quality and the various costs associated with controlling or mitigating vapor loss. Published data for solubility parameters is normally 25° C. unless stated otherwise. The hydrogen bonding solubility parameter is most changed with temperature. Equations for estimating the change in the solubility parameters with temperature predict that the solubility parameters of the blend will decrease with increased temperature and the sphere of solubility will become larger for the resin. This generally means solubility will improve with increased temperature. However, even if increasing temperature is effective to dissolve a resin, once the temperature returns to room temperature, the resin may precipitate, resulting in a cloudy lacquer.

Hence, one approach to finding a blend to dissolve CAB and create a clear top coat might be to use HSP's to calculate and minimize RED. This method was found to have some success for making a clear top coat lacquer only if the impact of the resin(s) and co-resin(s) percentages was taken into account empirically. In some experiments, although HSP predicted very good solubility, the resulting top coat was hazy. And, in some cases, HSP predicts a blend will not work as well as it actually does.

Some examples of false predictions using HSP theory were found for both commercial products and prepared samples. For example, the calculated RED for a solvent/plasticizer mixture reminiscent of Seche Vite top coat to dissolve CAB resin is 0.86 and 0.76 for a similar mixture without toluene. According to HSP theory, the solubility should increase with decreasing RED, yet numerous samples were made with RED well below the Seche Vite simulated solution and yet these solutions did not dissolve the CAB resins as well as the Seche Vite simulated solution.

According to the distributor/manufacturer of CAB, the haze is partly due to cellulose impurity which is measured as ash content and is reported as <0.05% on the technical data sheet provided. Further, the vendors recommended filtering out the impurity using a 1 μm filter. However, the haze is not removed with a 1 μm filter and the vendors theory also falls apart when you notice that the % haze varies appreciably when CAB is dissolved in different mixtures. Clearly, certain mixtures of solvents and plasticizers reduce the haze % below visible levels, which proves the haze is due to varying solubility of CAB resins in different solutions.

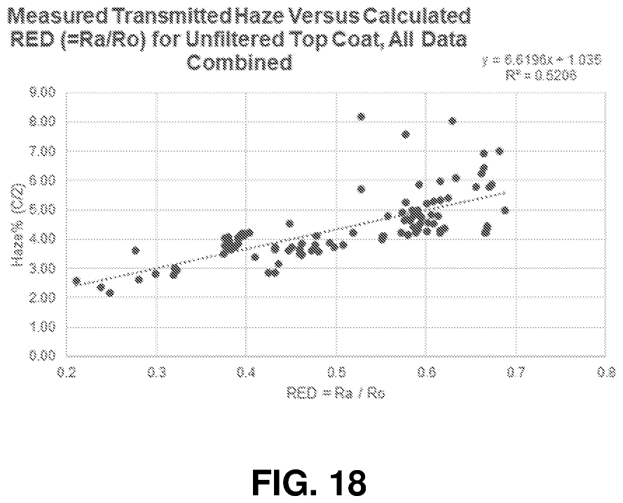

Referring to FIG. 18, a graph plotting % Haze(C/2) versus calculated Relative Energy Difference (RED) for unfiltered top coat samples is shown. This figure illustrates a correlation between the RED calculated from HSP's and the unfiltered top coat % Haze(C/2). As RED decreases for a given fixed resin wt %, the % Haze decreases. The scatter in the data pertains mainly to varying resin and co-resin wt %. Data suggests that in order to make a top coat that is clear with or without filtering, the RED and resin(s) wt % need to be below a certain threshold.

Based on the correlation observed in FIG. 18, a semi-empirical model was developed to predict the % Haze(C/2) of the unfiltered top coat from a known composition and the calculated RED between the resin and non-resin. The model takes the form shown in Equation 5 below:

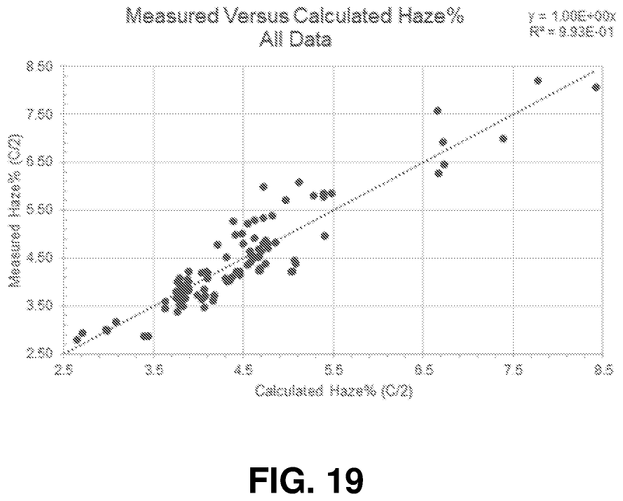

Haze %(C/2)=A*RED\hat{ }B+C*(Wt % CAB-381-0.5)+D*(Wt % CAB-551-0.2)+E*(Wt % CAB-381-2)+F*(Wt % Acrylates Copolymer) Equation 5:

Where, for all data combined (top coats with and without acrylates copolymer), the least squares fit is found with A=5.6824E+00; B=2.0018E+00; C=1.3312E−01; D=3.0268E−01; E=2.8480E−01; and F=1.7040E−02. The measured and calculated % Haze(C/2) are in good agreement generally (R\hat{ }2=0.88, R=0.94). The average absolute error for calculated % Haze (C/2) using this semi-empirical formula is 6.63%. The results of this curve fit are shown in FIG. 19, which is a plot of measured versus calculated % Haze(C/2) for all data.

With regards to grade selection of acrylates copolymer, it is important to note that some grades do not dissolve in ester solvents whereas others do. Others are meant for waterborne cosmetics systems at specific pH levels, such as a skin emollient, and are not applicable to a top coat composition. Furthermore, if not dissolved completely or properly, acrylates copolymer yielded hazy results.

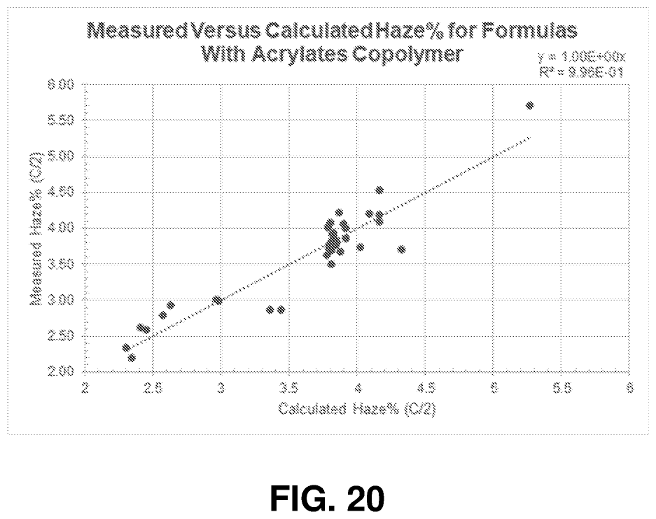

As FIG. 20 shows, less calculation error is generally found for the formulas with Acrylates Copolymer. For top coats with acrylates copolymer, the least squares fit is found with A=7.5899E+01; B=5.8134E+00; C=1.0985E−01; D=3.6694E−01; E=1.6139E−01; F=7.5308E−02. The measured and calculated Haze % are in good agreement generally (R\hat{ }2=0.87, R=0.93). The average absolute error for calculated % Haze(C/2) using this semi-empirical formula is 3.71%.

In both cases above (with and without acrylates copolymer) the coefficient F for acrylates copolymer is an order of magnitude smaller than the coefficients C, D, or E for the CAB grades. Also note that the coefficient C for CAB-381-0.5 is less than the coefficients D and E for the other CAB grades. Also note that the coefficient D for CAB-551-0.2 is higher than coefficient E for CAB-381-2.

In light of certain inadequacies of HSP theory to calculate a suitable blend for creating a clear top coat lacquer, in one or more embodiments, a formulation method of a clear top coat composition may involve a calculation of CAB solubility in the overall mixture using experimental solubility data. The solubility of CAB was determined in various solvents and plasticizers. It follows that the total CAB solubility (S) in a mixture of solvents and plasticizers is the sum of the individual solubilities (Sᵢ) and corresponding weight fraction (xᵢ), as shown below (and n is the number of solvents, plasticizers, co-resins, that CAB has some solubility in):

S=Σᵢ₌₁ⁿSᵢxᵢ Equation 6:

Based on the total solubility of CAB in the solvent/plasticizer mixture above, a maximum soluble wt % of CAB in said mixture may be determined by:

$$100 * ( \frac{S}{1 + S} ) \qquad Equation 7$$

In a further embodiment, an amount of CAB added to the mixture may be less than the maximum soluble wt % of CAB by some safety margin (Δ, see Example 14, Table 22) in order to prevent haze from forming in the top coat bottle from, for instance, solvent losses by evaporation over time, the top coat mixture not mixing perfectly, or fluctuations in temperature. This formulation method has many inadequacies, but was useful in creating some clear top coat formulas.

It was shown that a preferred mixture to dissolve CAB comprises a butyrate ester, an acetate ester, an alcohol, and a component with high dispersion (and low polarity and low hydrogen bonding) to match the cellulose ring structure backbone. Additionally, each mixture component must be compatible with each other. Additional factors considered beyond solubility include evaporation rate, viscosity, surface tension, density, odor, Hansen Solubility Parameters, relative percentage of volatile and non-volatile components (i.e. dry % solids), acidity, color, flammability, and toxicity. Butyrate esters such as methyl and ethyl butyrate are Generally Recognized as Safe (GRAS) and are commonly used as flavoring and fragrance ingredients in food. Ethyl butyrate has an evaporation rate and viscosity similar to n-butyl acetate. Methyl butyrate has an evaporation rate in between butyl and ethyl acetate. Whereas ethyl acetate and n-Butyl Acetate are used ubiquitously in nail lacquer products, methyl and ethyl butyrate esters have not been utilized prior to this disclosure.

According to FIG. 2, the butyryl substitution on the average ideal CAB monomer is 46 mol %. Based on the high percentage of butyryl R groups in all grades of CAB, it would seem critical to incorporate butyrate esters in the solvent mixture. Functional top coat compositions are generally comprised of 0% to 80% butyrate ester by weight. A preferred composition would include 30% to 80% butyrate ester by weight to closely match the mol percentage of butyryl side groups on CAB and allow for additional solvents, plasticizers and other additives.

Additionally, a selection of acetate esters may be incorporated in a top coat composition including methyl acetate, ethyl acetate, n-butyl acetate, iso-butyl acetate, tert-butyl acetate, sec-butyl acetate, propyl acetate, and isopropyl acetate, to name the most common and those with exceptional volatility. The most desirable candidates to optimize clarity and other aspects of the top coat formula in solution may be ethyl acetate, n-butyl acetate, iso-butyl acetate, and propyl acetate. From FIG. 2, the acetyl substitution on the average ideal CAB monomer is 22 mol %. A working top coat composition may comprise 0% to 70% acetate ester by weight. A preferred solution may contain 0% to 15% acetate ester by weight to closely match the mol percentage of acetyl side groups on CAB and allow for additional solvents, plasticizers and other additives.

Various alcohols including isopropanol, ethyl alcohol, denatured alcohol (i.e. SDA40A or SDA40B), n-butanol, iso-butanol, tert-butanol, and sec-butanol may be used to increase compatibility of the solvent mixture with the hydroxyl R-groups on CAB. The preferred alcohol reagent may be ethanol, but other alcohols (or mixtures thereof) may produce a clear top coat. High molecular weight alcohols, such as benzyl alcohol and phenol may also be used, but usually evaporate very slowly and become part of the film. Refer to ASTM D3539-11 for Evaporative Rates of Volatile Materials. Certain alcohols may also help mask the odor of the formula. For instance, SDA40B contains a denaturant and has odor reminiscent to sun tan lotion and n-butanol is an alkanol with a low Odor Detection Threshold (ODT).

With respect to the ideal composition of alcohol to match the proportion of —OH groups on the average ideal CAB monomer, FIG. 2 indicates only 7 mol %. Accordingly, functional top coat nail lacquers can be prepared with 0% to 20% by weight alcohol. But, the preferred composition of alcohol is 2% to 4% by weight for formulas which contain acrylates copolymer as a co-resin with CAB as the main resin and that do not require filtering to achieve <=2.1% Haze(C/2). For formulas that contain only CAB as a resin, higher alcohol content in the range of 11% to 16% by weight is preferred. The addition of alcohol helps with CAB solubility, wetting of the CAB, increasing viscosity, improved spreading and control of evaporation rate.

According to FIG. 2, the cellulose backbone on the average ideal CAB monomer comprises 25 mol %. A molecule with a phenyl group such as toluene, which has high dispersion and low polarity and hydrogen bonding HSP's (toluene HSP's: δ_{d}=18, δₚ=1.4, δₕ=2, where HSP units are MPa^{1/2}) is very compatible with the cellulose backbone. This is partly evidenced by the Seche Vite top coat which contains about 30% weight toluene and results in a clear top coat lacquer. It is also evidenced by the high dispersion HSP of cellulose (cellulose HSP's: δ_{d}=24.3, δₚ=19.9, δₕ=22.5, where HSP units are MPa^{1/2}).

Triphenyl Phosphate (TPP) is a plasticizer which contains 3 phenyl groups per mole and has high dispersion and low polarity and hydrogen bonding HSP's. If the ideal phenyl group percentage in a mixture to dissolve the ideal CAB is 25 mol %, then the ideal TPP percentage is 25/3 mol % (or 8 mol %). Although TPP is a preferred plasticizer for its ability to help dissolve CAB, it is also suspected of being an endocrine disruptor. Although TPP is often used in nail lacquers, it is included in some listings of the top 7 ingredients to avoid in cosmetics products.

Hence, a range of other viable plasticizers can be used including Trimethylpentanediyl dibenzoate; Trimethyl pentanyl diisobutyrate; Propylene carbonate; Acetyl tributyl citrate; Sucrose acetate isobutyrate; and Adipic acid/Neopentyl glycol/Trimellitic anhydride copolymer; triethyl citrate; tributyl citrate; epoxidized soybean oil and other natural oils of plant origin; and triacetin. Although phthalates such as diethyl phthalate, dibutyl phthalate, and butyl benzyl phthalate may function as a plasticizer, they are not preferred due to the classification of phthalates as known endocrine disruptors. Additionally, certain ingredients may act (at least partially) as a plasticizer in a formula, such as ethyl butyrate, dimethicone, certain fragrance ingredients, and the low viscosity CAB grades like CAB-551-0.2, but they are not counted/classified as plasticizer for the purpose of defining the wt % of plasticizer herein.

Each plasticizer addition level effects other properties of the top coat formula, such as viscosity, adhesion, hardness, gloss and flexibility. Although an ideal total concentration range for the given plasticizers in a top coat composition may be between 0% and 20% by weight, a more acceptable weight percent range is between 0% and 10% by weight. The preferred weight percentage of plasticizers is more narrowly defined between 1% and 4% by weight. And, the preferred plasticizers are Trimethylpentanediyl Dibenzoate, Trimethyl pentanyl diisobutyrate, and Propylene Carbonate and mixtures thereof.

Various CAB grades including (but not limited to) CAB-381-2, CAB 381-0.5, and CAB-551-0.2 can be used together at varying weight percentages to produce a CAB composition with distinct viscosity, adhesive, and mechanical properties. To produce an ideal top coat, the CAB concentration should be between 6% to 20% by weight. The CAB concentration is then ideally constrained to optimize dissolution by solvents, thereby improving the clarity of the top coat. This ideal % may vary depending on whether a co-resins is used, the chosen grade(s) of CAB, and the levels of various other solvents, plasticizers and additives. For instance, the ideal CAB % is narrowly defined between 8% to 12% by weight for formulas with a co-resin and that do not require filtering to achieve % Haze(C/2)<=2.1%. For formulas without a co-resin and that do require filtering to achieve good clarity, the ideal CAB % is higher: 16% to 20% by weight.

In one or more embodiments, clear nail top coat compositions as described above may also comprise one or more additives. For example, sucrose benzoate may be a suitable additive because it “imparts good film hardness, excellent gloss, and depth of gloss” (Lanxess Uniplex 280CG) and is readily soluble in the solvents described. Other additives which may improve gloss and are compatible with a clear nail lacquer top coat are certain polysiloxanes (such as dimethicone, diphenyl dimethicone and phenyl trimethicone), diacetone alcohol and certain co-resins such as tosylamide/epoxy resin, acrylates copolymer and nitrocellulose. Other potential additives may include UV inhibitors such as benzophenone-1 and etocrylene; colorants and fragrances. The preferred other additives are sucrose benzoate, dimethicone, acrylates copolymer, fragrance, and benzophenone-1. Similar to CAB, there are many grades of nitrocellulose available which can be used, but RS ½ Sec and RS ¼ are the most popular due to their viscosity and mechanical properties when applied to nails. Additionally, dimethicone is available in a variety of viscosity grades; dimethicone having a kinematic viscosity of 5-10 mm²/s is preferable for cosmetics and the embodiments demonstrated herein. An ingredient alike dimethicone is Citropol® 1A, a biopolymer, Foley, P. and Yang, Y. (U.S. Pat. No. 10,059,801).

Compared to the prior art, in which colorant added is used to mask cloudiness, the present invention permits the measured addition of colorant without requiring it as a mask nor compromising the clarity of the top coat. As such, the development of varieties of clear, dyed, colored, or pigmented nail lacquer or top coat formulation products are within the scope of the embodiments described herein.

Top coat nail lacquers typically have a fruity chemical solvent smell. The fruity smell is typically due to ethyl and butyl acetate. Certain plasticizers and colorants can also impart a fruity odor, such as TCE and Violet #2. In some products, the presence of alcohol can be smelled. In the case of Seche Vite, toluene imparts a strong chemical smell reminiscent of model glue. Butyrate esters impart a strong fruity smell to a top coat. Many cosmetics may obtain ingredients to control odor, such as a fragrance or masking agent. A typical fragrance is composed of a mixture of ingredients which evaporate at different rates and tell a story for the product. The top note fragrance ingredients are those that evaporate the fastest and typically have a strong smell. The middle note fragrance ingredients evaporate slower than the top note ingredients but faster than the base note ingredients. The base note ingredients evaporate slowly and impart a lasting odor to the product. While the Relative Evaporation Rate (R) or vapor pressure is used to characterize the rate at which a fragrance ingredient evaporates, the Odor Detection Threshold (ODT) is used to characterize how strong an odor is. Ingredients with low ODT can be smelled at low vapor concentrations relative to those with high ODT. Hence, ingredients with low ODT can mask ingredients with high ODT, especially if they have similar evaporation rates. The Odor Intensity (OI) increases proportionally with increasing concentration of an ingredient and decreasing ODT. Table 4 below provides odor data for various compounds.

TABLE 4
Odor data for various chemicals.

| Chemical | ODT (ppb) | R | Pᵥ*(mmHg) |
|---|---|---|---|
| Methyl Butyrate | 60 to 76 | 2.3 | 25 |
| Ethyl Butyrate | 1 | 0.9 | 8 |
| Iso-Butyl Acetate | 66 | 1.46 | 14 |
| Ethyl Acetate | 3900 | 4.1 | 112 |
| Ethanol | 100000 | 1.7 | 44.6 |
| Toluene | 2000 | 2 | 28.4 |
| n-Butyl Acetate | 66 | 1 | 11.5 |
| Methyl Propionate | — | 5.3 | 87.56 |
| Ethyl Propionate | — | 2.6 | 37.62 |

The concentration of different butyrate esters in the solvent mixture may be manipulated to mask certain odors and manipulate tack-free time. For example, a concentration of ethyl butyrate may be higher than that of methyl butyrate, or in another example, acetate esters with a similar evaporation rate to methyl/ethyl butyrate may also be used. Propionate (or propanoate) esters such as methyl or ethyl propionate are also good masking fragrance ingredients with low ODT and an evaporation rate which could help mask the top note. Lastly a wide array of fragrances (or fragrance ingredients) may be added to the top coat which will have a pleasant odor and mask other less desirable solvent odors.

While reflectance and transmittance can be used as a quantitative indicator of clarity, an accompanying visual assay can provide important observational data relevant to consumer interests regarding a top coat's clarity based on the degree of haze in material using light transmittance through the sample. A typical barrier of entry for top coat and nail polish cosmetics is the visual nature of the product. Consumers who observe distinct cloudiness or clumps of solute in their top coat formula may very likely choose a clearer formula in lieu of the hazier brand.

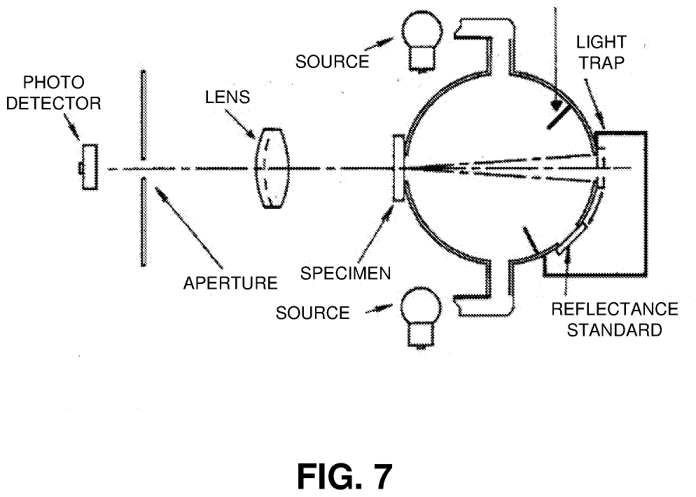

Haze is caused by the scattering of light during reflection and transmission through a given material. Reflective haze occurs when light is reflected at narrow-to-wide angles from a material's surface giving the substance a milky or hazy quality. Reflective haze measurement was administered using Rhopoint IQ Haze Meter and according to ASTM E430. Transmission haze occurs as a result of light scattering as it passes through an object and is based on the material's refractive index, the concentration of dispersed particles, contaminants, and air spaces in solution. Transmission haze % measurement is outlined by ASTM D1003-13 as the ratio (diffuse transmittance)/(total transmittance)*100. Referring to FIG. 7, a diagram of an exemplary % Haze(C/2) measurement device in accordance with ASTM D1003-13 is shown. Unlike haze, which is seen at various angles, clarity (i.e., transparency) is a measurement of the linearity of a light beam shining perpendicularly through a sample. Therefore, a valuable test to measure a top coat's clarity holistically would include a qualitative assessment for relative transmittance compared to a clear standard as well as a quantitative assessment using a spectrophotometer or goniospectrophotometer (in the case of reflected haze) to determine the amount of light scattered when passing through the sample. Taking a replicable qualitative measurement may also account for the longer length by which light passes through the glass of a nail polish bottle compared to, e.g., a 1 cm cuvette typically used in a spectrophotometer. Wider cuvettes (e.g., 20 or 30 mm) may more closely approximate the thick wall of a nail polish bottle (e.g., 33 mm). Even so, cuvettes are typically made from a different material or grade of glass than that of a nail polish bottle.

A method of filtering top coats was found which reduces the Haze % (C/2), with relative effectiveness impacted by filter pore size and the presence or absence of certain ingredients in the top coat formula which act as filtration aids.

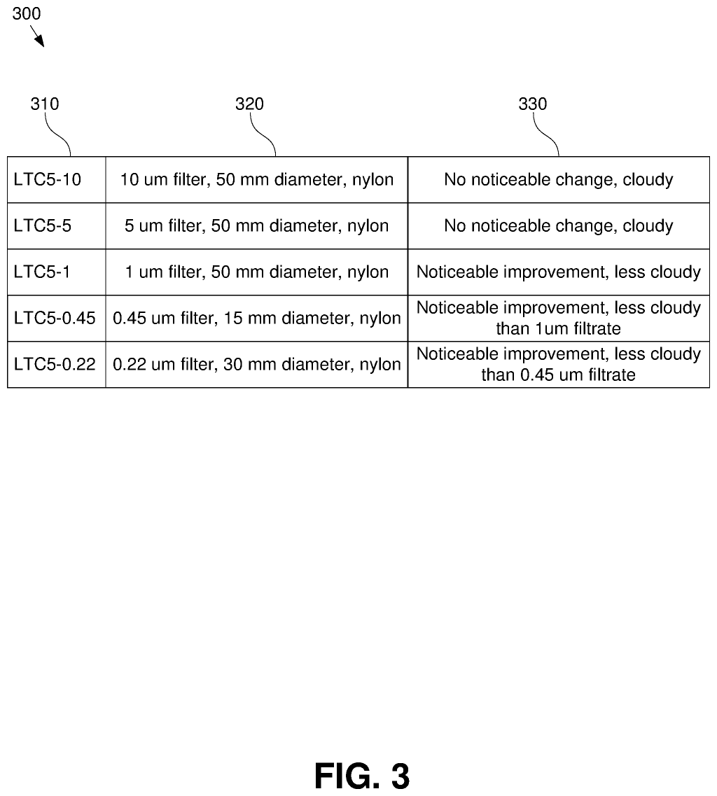

A membrane filter pore size rating of 0.22 μm was found to be very effective (See FIG. 3). Secondly, the filtration does not have a deleterious effect on the mechanical and chemical properties. Smaller filter pore size than 0.22 μm may reduce haze further, but at the cost of lower flowrate and higher pressure drop. High pressure drop may cause flashing of volatile, flammable vapors, which may present a safety hazard as well as change the composition and properties of the formula. Similarly, vacuum filtration is not preferred because it may cause solvents with high vapor pressure or low boiling point to volatilize.

A common filtration aid used in industry is diatomaceous earth (DE). In practice, the method is employed with large industrial filters (filter presses, basket filter centrifuges, rotary drum filters, etc . . . ). In this method, the DE is precoated onto the filter cloth/membrane using a separate body feed solution. Then the product to be filtered is pumped or vacuumed across the DE coated filter. The DE precoat often improves filtration results significantly. However, DE is not a desired ingredient in nail lacquer products, even as an impurity, since it contains crystalline silica, which may have traces of asbestos, which is a known carcinogen. Also, DE particles would not be stable in suspension in a nail lacquer top coat. Although more benign filter aids than DE exist, such as wood cellulose and perlite, a benefit of the current invention is that numerous top coat ingredients which are safe and stable in a nail top coat lacquer act as filtration aids. Namely, the butyrate esters, diphenyl dimethicone, and co-resins tosylamide/epoxy resin and acrylates copolymer were all found to improve the percentage of haze which is filtered out.

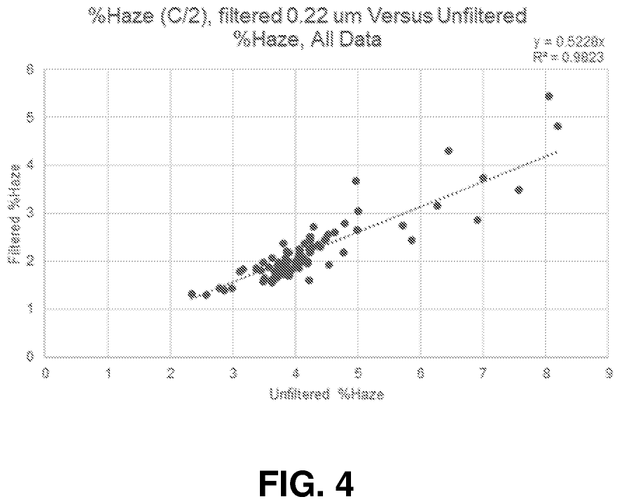

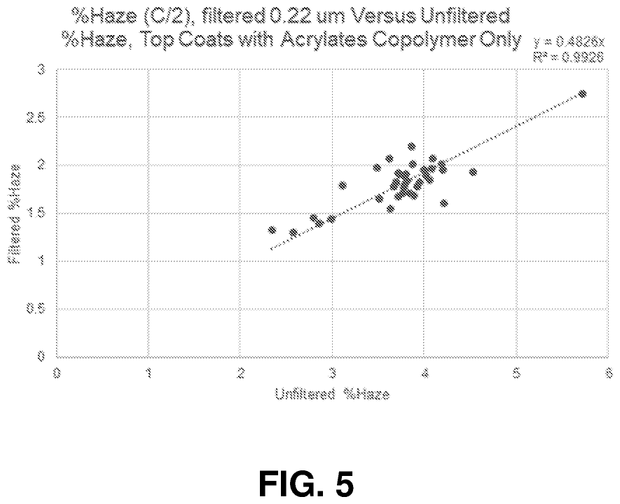

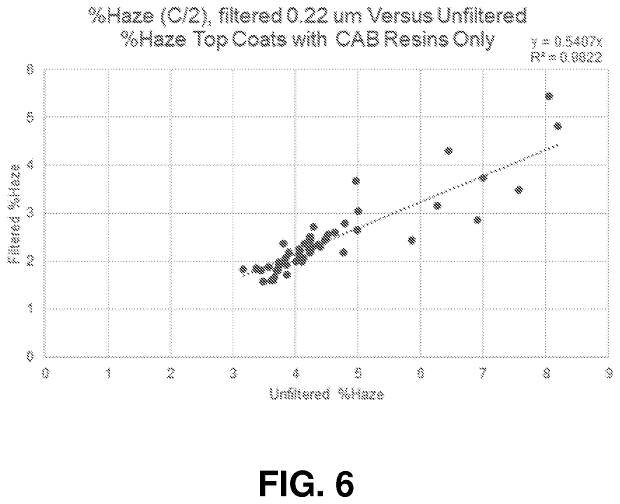

FIGS. 4 through 6 show the results of passing numerous different top coat samples through a 47 mm, circular, in-line, 0.22 um, polypropylene, membrane filter. FIG. 4, which is for all CAB based top coat data combined with and without acrylates copolymer co-resin, shows an average of 48% reduction in % Haze(C/2) from filtering. FIG. 5, which is for all CAB based top coat data with acrylates copolymer co-resin, shows an average of 52% reduction in % Haze(C/2) from filtering. And lastly, FIG. 6, which is for all CAB based top coat data without acrylates copolymer co-resin, shows an average of 46% reduction in % Haze(C/2) from filtering. Comparing FIGS. 5 and 6, the % decrease in % haze(C/2) is about 6% more when top coats containing CAB and acrylates copolymer are filtered, than top coats with CAB resin only.

Exemplary formulations, their constituents, measurement results, and procedural methods are tabulated and discussed below.

### Example #1

Preliminary top coats (some reminiscent of Seche Vite) were made according to the composition given in the table and procedure described below where toluene is replaced with ethyl acetate in various proportions, along with varying amounts of dimethicone and other ingredients.

TABLE 5
Preliminary testing of top coats with and without toluene.

| | | | | Wt % Formula | | | |
|---|---|---|---|---|---|---|---|
| | | Other | | TC- | TC- | TC- | TC- |
| Part | INCI Name | Name | Mfg. | P-3 | P-4 | P-4A | P-5 |
| A | Butyl Acetate | nBA | | 38.00 | 37.85 | 37.75 | 38.08 |
| | Toluene | | | 0 | 31.08 | 31.00 | 15.47 |
| | Ethyl Acetate | | | 31.10 | 0 | 0 | 15.56 |
| | Cellulose Acetate | CAB- | Eastman | 17.06 | 17.07 | 17.03 | 17.06 |
| | Butyrate | 381-0.5 | | | | | |
| | Cellulose Acetate | CAB- | Eastman | 2.45 | 2.43 | 2.43 | 2.47 |
| | Butyrate | 551-0.2 | | | | | |
| B | Isopropyl Alcohol | IPA | | 7.53 | 7.71 | 7.70 | 7.51 |
| | Trimethyl Pentanyl | TXIB | Eastman | 3.87 | 3.85 | 3.84 | 3.86 |
| | Diisobutyrate | | | | | | |
| | Dimethicone | PMX-200, | Dow | 0 | 0 | 0.25 | 0 |
| | | 10 CSt | Corning | | | | |

| | | | | |
|---|---|---|---|---|
| Total: | 100 | 100 | 100 | 100 |
| Haze % (C/2): | 4.2 | 2.0 | 2.0 | 2.6 |

The samples in Table 5 above were made according to the procedure below:
a) Weigh out and combine the Part A CAB's and set aside.
b) Weigh out and combine the Part A solvents in the desired container.
c) Set the mixer for 690 RPM and slowly add the CAB's to the Part A solvents.
d) Increase the speed to 1610 RPM and mix for 20 min.
e) Weigh out and combine the Part B ingredients.
f) Set the mixer to 690 RPM and add the Part B ingredients to Part A and mix for 20 mins.

These tests were conducted with 400 gr total sample size and mixed with a small diameter saw tooth impeller. A glass beaker was used for samples with toluene and HDPE cups were used for the other samples.

The samples TC-P-4, TC-P-4A and TC-P-5 were much clearer according to the Haze % results in Table 5 than the other sample (TC-P-3) in which toluene was replaced with ethyl acetate. Also, the Haze % increased when toluene concentration was decreased from 31% to 15%. Dimethicone was found to make the dry nail top coat lacquer surface smooth, but had no measurable impact on haze.

### Example #2

A batch of top coat samples (LTC-1/2/3/4/5) was made without toluene according to the composition given in the table and procedure described below, where toluene is replaced with ethyl acetate, n-butyl acetate, n-propyl acetate and IPA in various proportions. Various chemical/material properties were measured and compared with Seche Vite.

TABLE 6
Top coat without toluene (Seche Vite Composition estimated below, balance ~30.9
Wt % toluene).

| | | | | Wt % Formula | | | | | |
|---|---|---|---|---|---|---|---|---|---|
| | | Other | | LTC- | LTC- | LTC- | LTC- | LTC- | Seche |
| Part | INCI Name | Name | Mfg. | 1 | 2 | 3 | 4 | 5 | Vite |
| A | Benzo- | B1 | Sigma | 0.10 | 0.10 | 0.10 | 0.10 | 0.10 | 0.10 |
| | phenone-1 | | | | | | | | |
| | Dimethicone | PMX-200, | Dow | 0.30 | 0.30 | 0.30 | 0 | 0 | 0 |
| | | 10 CSt | Corning | | | | | | |
| | Trimethyl | TXIB | Eastman | 2.29 | 2.29 | 2.29 | 1.36 | 1.36 | 1.5 |
| | Pentanyl | | | | | | | | |
| | Diisobutyrate | | | | | | | | |
| | Butyl Acetate | nBA | | 51.30 | 38.23 | 39.81 | 40.33 | 38.73 | 37 |
| | Isopropyl | IPA | | 15.75 | 7.57 | 15.91 | 16.11 | 7.67 | 10 |
| | Alcohol | | | | | | | | |
| | Propyl | nPA | | 0 | 0 | 4.97 | 5.03 | 0 | 0 |
| | Acetate | | | | | | | | |
| | Ethyl Acetate | EA | | 10.04 | 31.29 | 16.40 | 16.62 | 31.70 | 0 |
| B | Cellulose | CAB- | Eastman | 15.38 | 15.38 | 15.38 | 12.16 | 12.16 | 19.5 |
| | Acetate | 381-0.5 | | | | | | | |
| | Butyrate | | | | | | | | |
| | Cellulose | CAB- | Eastman | 2.99 | 2.99 | 2.99 | 4.20 | 4.20 | 1 |
| | Acetate | 551-0.2 | | | | | | | |
| | Butyrate | | | | | | | | |
| | Cellulose | CAB- | Eastman | 1.85 | 1.85 | 1.85 | 4.10 | 4.10 | 0 |
| | Acetate | 381-2 | | | | | | | |
| | Butyrate | | | | | | | | |

| | | | | | | |
|---|---|---|---|---|---|---|
| Total: | 100 | 100 | 100 | 100 | 100 | 69.1 |
| (density in g/cc measured at room | 1.008 | 1.006 | 0.991 | 0.991 | 1.006 | 0.994 |
| temperature ~21*C) S.G. | | | | | | |
| (Calculated from HSP’s) RED | 0.78 | 0.75 | 0.77 | 0.77 | 0.75 | 0.87 |
| % Solids | 22.91 | 22.91 | 22.91 | 22.91 | 22.91 | 21.83 |
| (Relative evaporation rate to reach 90% | 0.85 | 1.66 | 2.79 | 2.79 | 1.66 | 1.44 |
| dry film, R of nBA = 1) R 90% | | | | | | |
| (Calculated) Flash Point (*C) | 18.2 | 12 | 13.6 | 13.7 | 12.1 | 11.5 |
| Viscosity (mPa-s, 20*C) | 992 | 897 | 913 | 1048 | 1024 | 967 |
| Surface Tension (Dyne/cm) | 45 | 45 | 45 | 52 | 52 | 31 |
| Transmitted % Haze (C/2) | 6.2 | 6.1 | 5.6 | 6.8 | 6.7 | 2.1 |
| 20* Gloss | 71.2 | 71.8 | 72.8 | 71.4 | 73 | 77.9 |
| (Reported in LogHc units, lower | 323 | 342 | 315 | 332 | 356 | 324 |
| values indicate film is less | | | | | | |
| hazy) Reflected Haze | | | | | | |
| Hardness BHN 500 g | 3.84 | 3.8 | 3.9 | 3.84 | 3.84 | 3.29 |
| Pencil Hardness | HB | HB | HB | HB | HB | 2B |
| 3 min Pull Off (mJ) | 1.53 | 1.53 | 1.53 | 1.61 | 1.61 | 1.38 |
| 5 min Pull Off (mJ) | 1.7 | 1.7 | 1.7 | 2.18 | 2.18 | 1.99 |
| (smaller number →smoother surface) | 1.86 | 1.86 | 1.86 | 1.86 | 1.86 | 1.86 |
| Coefficient of Sliding Friction | | | | | | |
| (N-m) Impact/Flex | 0.42 | 0.42 | 0.42 | 0.42 | 0.42 | 0.28 |
| (from liquid to dry film) Shrinkage % | 77 | 77 | 77 | 78 | 78 | 82 |

The samples in table 6 above were made according to the procedure below:
a) Weigh out and combine the Part A ingredients in a 400 ml HDPE pint cup in the order provided and set aside with the lid closed to prevent evaporation.
b) Weigh out and combine the Part B CAB's in a 400 ml HDPE pint cup in the order provided and set aside with the lid closed.
c) Using the mixer with the 2″ 3-blade propeller, begin mixing part A at 500 RPM and then slowly add the Part B ingredients over a period of 3-4 minutes. The CAB must be added slowly to prevent large clumps from forming which take a longer time to dissolve. After 6 minutes of mixing close and seal the lid and allow the solution to rest overnight. The next morning, mix the solution for another 15 mins at 500 RPM with the same mixer/impeller.

Sample LTC-3 was the best sample in this LTC set of experiments based on measured properties and wear testing. The main need for improvement is the % Haze(C/2) is more than double that of Seche Vite. Note that despite the fact the calculated RED is lower than Seche Vite for all the LTC samples, the % Haze(C/2) is more than double that of Seche Vite. Other properties of the LTC samples are comparable or better than Seche Vite.

### Example #3

Clear top coats A1, A2, and A3 were made without toluene according to the composition given in the table and procedure described below, where toluene is replaced with various acetates, alcohols, butyrates, plasticizers and other additives in various proportions. Various chemical/material properties were measured and compared with Seche Vite.

TABLE 7
Various clear topcoats compared with Seche Vite, LVX Gel, and LTC-3 Top Coat.

| | | | | Wt % Formula | | | | | |
|---|---|---|---|---|---|---|---|---|---|
| | | Other | | | | | LTC- | LVX | Seche |
| Part | INCI Name | Name | Mfg. | A1 | A2 | A3 | 3 | Gel | Vite |
| A | Trimethylpentanediyl | Benzoflex | Eastman | 0.76 | 0.78 | 0.75 | See Example #2 for | | |
| | Dibenzoate | 354 | | | | | LTC-3 and Seche Vite. | | |
| | Diphenyl Dimethicone | KF54-HV | Shin-Etsu | 0 | 0 | 0.31 | See ingredients listed | | |
| | Trimethyl Pentanyl | TXIB | Eastman | 0 | 0.39 | 0 | for LVX Gel in | | |
| | Diisobutyrate | | | | | | “Background of | | |
| | Ethyl Butyrate | EB | | 26.69 | 25.70 | 25.42 | Invention” section | | |
| | Isopropyl Alcohol | IPA | | 2.5 | 2.14 | 2.39 | | | |
| | SDA40B | | | 0.83 | 0.64 | 0.79 | | | |
| | Iso-Butyl Acetate | iBA | | 1.16 | 2.99 | 1.11 | | | |
| | Methyl Butyrate | MB | | 15.72 | 14.92 | 14.92 | | | |
| | Ethyl Acetate | EA | | 26.94 | 24.19 | 25.64 | | | |
| B | Benzophenone-1 | B1 | Sigma | 0.10 | 0.10 | 0.10 | | | |
| | Sucrose Benzoate | UNIPLEX | LANXESS | 1.97 | 2.55 | 2.98 | | | |
| | | 280CG | | | | | | | |
| | Triphenyl Phosphate | TPP | | 6.33 | 8.46 | 8.80 | | | |
| C | Cellulose Acetate | CAB-381- | Eastman | 5.61 | 1.37 | 1.05 | | | |
| | Butyrate | 2 | | | | | | | |
| | Cellulose Acetate | CAB-381- | Eastman | 9.93 | 13.24 | 13.16 | | | |
| | Butyrate | 0.5 | | | | | | | |
| | Cellulose Acetate | CAB-551- | Eastman | 1.46 | 2.52 | 2.59 | | | |
| | Butyrate | 0.2 | | | | | | | |

| | | | | | | |
|---|---|---|---|---|---|---|
| Total: | 100 | 100 | 100 | | | |
| (density in g/cc calculated or measured at room | 1.03 | 1.06 | 1.06 | 0.991 | ND | 0.994 |
| temperature ~21*C) S.G. | | | | | | |
| (Calculated from HSP's) RED | 0.56 | 0.56 | 0.55 | 0.77 | — | 0.87 |
| (Filtered) % Solids | 28 | 31.44 | 31.52 | 22.91 | 37.15 | 21.83 |
| Tack-Free Time (min) | 2.5 | 2.5-3 | 2-2.5 | 2.5 | 2.5 | 3 |
| (Relative evaporation rate to reach 90% dry film, R of | 1.72 | 1.60 | 1.85 | 2.79 | — | 1.44 |
| nBA = 1) R 90% | | | | | | |
| (Calculated) Flash Point (*C) | 13.4 | 13.9 | 13.4 | 13.6 | — | 11.5 |
| Viscosity (mPa-s, 20*C) | 1024 | 1040 | 849 | 913 | 1056 | 967 |
| Surface Tension (Dyne/cm) | 42 | 59 | 61 | 45 | — | 31 |
| (Before filtering) Transmitted % Haze (C/2) | 4.4 | 3.9 | 2.9 | 5.6 | 2.1 | 2.1 |
| (After filtering) Transmitted % Haze (C/2) | 2.3 | 2.0 | 1.5 | 2.9 | — | — |
| 20* Gloss | 83 | 95 | 83 | 73 | 82 | 78 |
| (Reported in LogHc units, lower values indicate film is less | 300 | 275 | 275 | 315 | 271 | 324 |
| hazy) Reflected Haze | | | | | | |
| Hardness BHN 500 g | 2.2 | 1.2 | 0.9 | 3.9 | 1.2 | 3.29 |
| Pencil Hardness | 4B | 5B | 12B | HB | 2B | 2B |
| 3 min Pull Off (mJ) | 1.2 | 1.4 | 2.1 | 1.5 | 1.4 | 1.38 |
| 5 min Pull Off (mJ) | 1.9 | 2.4 | 1.8 | 1.7 | 2.1 | 1.99 |
| (smaller number → smoother surface) Coefficient of Sliding | 0.89 | 1.15 | 0.13 | 1.86 | 2.89 | 1.86 |
| Friction | | | | | | |
| (N-m) Impact/Flex | 0.14 | 0.14 | 0.14 | 0.42 | 0.14 | 0.28 |
| (from liquid to dry film) Shrinkage % | 77 | 70 | 68 | 77 | 70 | 82 |
| Tensile Strength (KPa) | 22 | 15 | 12 | 44 | Too | 50 |
| Elastic Modulus (MPa) | 0.48 | 0.12 | 0.06 | 0.31 | Brittle | 0.19 |
| | | | | | to | |
| | | | | | Test | |

The samples in Table 7 above were made according to the procedure below:
a) Weigh out the part B and C ingredients into separate weighing boats and set aside.
b) Weigh out and combine the Part A ingredients in the order shown into a 200 ml HDPE pint cup.
c) Using the mixer with the 1″ saw tooth impeller, begin mixing part A at 400 RPM and add the Benzophenone-1 and Sucrose Benzoate. Mix for a period of 4 minutes until the solids are completely dissolved.
d) Next, add the TPP and mix for another 4 mins until the solids are completely dissolved.
e) Next, begin slowly adding the CAB grades in the order shown while gradually increasing mixing speed from 400 RPM to 2000 RPM to maintain a vortex and prevent solids from floating and agglomerating on the surface. The CAB must be added slowly to prevent large clumps from forming which take a longer time to dissolve. It typically takes about 6 mins to add all the CAB grades and increase the mixing speed from 400 to 2000 RPM. Mix for 30 to 40 mins until all the CAB is dissolved and the viscosity and odor are acceptable.
f) Decrease mix speed to 200 RPM and mix for 5 mins to blend the top coat as it de-bubbles.
g) Filter the batch through a 0.22 μm filter using a peristaltic pump as follows:
   i) Rinse the tubing and in-line filter holder with 300-400 ml of n-butyl acetate at a speed of 20 RPM.
   ii) Take apart filter holder and wipe clean and allow tubing and filter holder to air dry for a few minutes.
   iii) Seat a 47 mm 0.22 μm PP filter membrane to the filter holder and wet the membrane with a few drops of n-Butyl Acetate. Seal the filter holder/membrane very tight to prevent leaks or bad filtering results. The gaskets must be seated to seal the filter.
   iv) Place the pump inlet tube in the mixed batch from step f and the pump outlet tube into a clean HDPE product container. The product container can be secured with a ring clamp fastened to a lab stand.
   v) Filter the batch at 4 RPM. When the filtering is almost complete, tilt the supply cup to get the remaining liquid. When there is no more supply liquid, continue to run the pump and let the tubing gravity drain down to the product container for several minutes until the tubing line is essentially cleared of remaining sample

The top coats A1 to A3 have better gloss than the previous top coats. Many other chemical, mechanical and optical properties which relate to the quality of the product, (such as viscosity, drying rate, hardness, spreading, shrinkage, smoothness and so on) were measured and found to compare very well with Seche Vite and LVX Gel Top Coat. Samples A2 and A3 also have better reflective haze and clarity (lower transmitted haze) than Seche Vite. Sample A1 has comparable spreading and mechanical properties to Seche Vite.

### Example #4

Clear top coats B2 and C2 were made without toluene according to the composition given in the table and procedure described below, where toluene is replaced with various acetates, alcohols, butyrates, plasticizers and other additives in various proportions. Sample B2 includes the co-resin Acrylates Copolymer and sample C2 includes co-resins Acrylates Copolymer and Nitrocellulose. Various chemical/material properties were measured and compared with Seche Vite and LVX Gel top coats.

TABLE 8
Clear topcoats with co-resins compared with Seche Vite and LVX Gel Top Coats.

| | | | | Wt % Formula | | | | | |
|---|---|---|---|---|---|---|---|---|---|
| | | | | | | | LTC- | LVX | Seche |
| Part | INCI Name | Other Name | Mfg. | B2 | A2 | C2 | 3 | Gel | Vite |
| A | Trimethylpentanediyi | Benzoflex | Eastman | 0.40 | 0.78 | 3.22 | See Example #2 for | | |
| | Dibenzoate | 354 | | | | | LTC3 and Seche Vite. | | |
| | Trimethyl Pentanyl | TXIB | Eastman | 1.11 | 0.39 | 1.44 | See ingredients listed | | |
| | Diisobutyrate | | | | | | for LVX Gel in | | |
| | Ethyl Butyrate | EB | | 15.11 | 25.70 | 12.91 | “Background of | | |
| | Butyl Acetate | nBA | | 0 | 0 | 0.72 | Invention” section | | |
| | Acrylates Copolymer | Pecorez | Phoenix | 20.19 | 0 | 3.37 | | | |
| | And Butyl Acetate | AC50 | | | | | | | |
| | | (50.19% | | | | | | | |
| | | resin in | | | | | | | |
| | | nBA) | | | | | | | |
| | Acrylates Copolymer | Avalure | Lubrizol | 0 | 0 | 12.69 | | | |
| | And Butyl Acetate | AC210 | | | | | | | |
| | | (74.74% | | | | | | | |
| | | resin in | | | | | | | |
| | | nBA) | | | | | | | |
| | Isopropyl Alcohol | IPA | | 1.83 | 2.14 | 0.23 | | | |
| | SDA40B | | | 0.66 | 0.64 | 0 | | | |
| | Iso-Butyl Acetate | iBA | | 1.18 | 2.99 | 1.01 | | | |
| | Ethyl Acetate And | (34% NC | Alchemix | 0 | 0 | 25.75 | | | |
| | Nitrocellulose And | resin: 51.86% | | | | | | | |
| | Isopropyl Alcohol | EA:IPA), ¼ | | | | | | | |
| | | s NC | | | | | | | |
| | Methyl Butyrate | MB | | 9.24 | 14.92 | 4.73 | | | |
| | *drops of 1% D&C | | | 0 | 0 | 0.41 | | | |
| | Violet 2 | | | | | | | | |
| | Ethyl Acetate | EA | | 26.44 | 24.19 | 21.81 | | | |
| B | Benzophenone-1 | B1 | Sigma | 0.10 | 0.10 | 0.10 | | | |
| | Sucrose Benzoate | UNIPLEX | LANXESS | 2.06 | 2.55 | 1.90 | | | |
| | | 280CG | | | | | | | |
| | Triphenyl Phosphate | TPP | | 7.05 | 8.46 | 3.24 | | | |
| C | Cellulose Acetate | CAB-381-2 | Eastman | 0.51 | 1.37 | 1.10 | | | |
| | Butyrate | | | | | | | | |
| | Cellulose Acetate | CAB-381- | Eastman | 10.05 | 13.24 | 4.79 | | | |
| | Butyrate | 0.5 | | | | | | | |
| | Cellulose Acetate | CAB-551- | Eastman | 3.58 | 2.52 | 0.47 | | | |
| | Butyrate | 0.2 | | | | | | | |
| D | Fragrance | | | 0.50 | 0 | 0.50 | | | |

| Total: | 100 | 100 | 100 | | | |
|---|---|---|---|---|---|---|
| (density in g/cc calculated or measured at room temperature | 1.04 | 1.06 | 0.995 | 0.991 | ND | 0.994 |
| ~21*C) S.G. | | | | | | |
| (Calculated from HSP's) RED | 0.37 | 0.56 | 0.54 | 0.77 | — | 0.87 |
| (Filtered) % Solids | 34.90 | 31.44 | 36.17 | 22.91 | 37.15 | 21.83 |
| Tack-Free Time (min) | 2.5 | 2.5-3 | 2.5 | 2.5 | 2.5 | 3 |
| (Relative evaporation rate to reach 90% dry film, R of | 3.63 | 1.60 | — | 2.79 | — | 1.44 |
| nBA = 1) R 90% | | | | | | |
| (Calculated) Flash Point (*C) | 13.9 | 13.9 | 10.7 | 13.6 | — | 11.5 |
| Viscosity (mPa-s, 20*C) | 913 | 1040 | 857 | 913 | 1056 | 967 |
| Surface Tension (Dyne/cm) | 39 | 59 | 45 | 45 | - | 31 |
| (Before filtering or As Received) Transmitted %Haze | 3.5 | 3.9 | 2.9 | 5.6 | 2.1 | 2.1 |
| (C/2) | | | | | | |
| (After filtering) Transmitted % Haze (C/2) | 1.8 | 2.0 | 1.5 | 2.9 | — | — |
| 20* Gloss | 94 | 95 | 92 | 73 | 82 | 78 |
| (Reported in LogHc units, lower values indicate film is less | 248 | 275 | 309 | 315 | 271 | 324 |
| hazy) Reflected Haze | | | | | | |
| Hardness BHN 500 g | 0.9 | 1.2 | 0.8 | 3.9 | 1.2 | 3.29 |
| Pencil Hardness | 5B | 5B | B | HB | 2B | 2B |
| 3 min Pull Off (mJ) | 1.5 | 1.4 | 1.5 | 1.5 | 1.4 | 1.38 |
| 5 min Pull Off (mJ) | 2.2 | 2.4 | 1.7 | 1.7 | 2.1 | 1.99 |
| (smaller number → smoother surface) Coefficient of Sliding | 1.33 | 1.15 | 1.68 | 1.86 | 2.89 | 1.86 |
| Friction | | | | | | |
| (N-m) Impact/Flex | <0.14 | 0.14 | 0.14 | 0.42 | 0.14 | 0.28 |
| (from liquid to dry film) Shrinkage % | 75 | 70 | 66 | 77 | 70 | 82 |
| Tensile Strength (KPa) | 11 | 15 | 15 | 44 | Too | 50 |
| | | | | | Brittle | |
| | | | | | to | |
| Elastic Modulus (MPa) | 0.06 | 0.12 | 0.08 | 0.31 | Test | 0.19 |

*1% Violet 2 added as a fraction of 1 drop by diluting 1 drop of 1% violet 2 in 10 gr of ethyl acetate and adding 4.1 gr of the mixture to the batch.

The samples in Table 8 above were made according to the procedure below:
a) Weigh out the part B and C ingredients into separate weighing boats and set aside.
b) Weigh out and combine the Part A ingredients in the order shown into a 200 ml HDPE pint cup.
c) Using the mixer with a 1″ saw tooth impeller, begin mixing part A at 400 RPM and add the Benzophenone-1 and Sucrose Benzoate. Mix for a period of 4 minutes until the solids are completely dissolved.
d) Next, add the TPP and mix for another 4 mins until the solids are completely dissolved.
e) Next, begin slowly adding the CAB grades in the order shown while gradually increasing mixing speed from 400 RPM to 2000 RPM to maintain a vortex and prevent solids from floating and agglomerating on the surface. The CAB must be added slowly to prevent large clumps from forming which take a longer time to dissolve. It typically takes about 6 mins to add all the CAB grades and increase the mixing speed from 400 to 2000 RPM. Mix for 17 mins.
f) Add the fragrance (part D) and mix an additional 3 mins at 2000 RPM.
g) Decrease mix speed to 200 RPM and mix for 5 mins to blend the top coat as it de-bubbles.
h) Filter the batch according to Example 3.

The co-resins Acrylates Copolymer and Nitrocellulose are very low haze, which helps with the clarity of the liquid, gloss, and thickness. Incorporating Acrylates Copolymer also reduces the amount of CAB required in the formula. Top coats B2 and C2 have better clarity, gloss, tack-free dry time, thickness, surface smoothness, and shrinkage than Seche Vite.

### Example #5

Clear top coats TC-1-F through TC-7-F were made without toluene according to the composition given in the table and procedure described below, where toluene is replaced with various acetates, alcohols, butyrates, plasticizers and other additives in various proportions. Samples TC-1-F to TC-4-F include the co-resin Acrylates Copolymer, while the remaining samples use CAB as the only resin. Various chemical/material properties were measured and included in Table 9 below.

TABLE 9
Clear topcoats with and without Acrylates Copolymer co-resin.

| | | | | Wt % Formula | | | | | | |
|---|---|---|---|---|---|---|---|---|---|---|
| | | Other | | TC- | TC- | TC- | TC- | TC- | TC- | TC- |
| Part | INCI Name | Name | Mfg. | 1-F | 2-F | 3-F | 4-F | 5-F | 6-F | 7-F |
| A | Dimethicone | PMX200, | DOW- | 0.29 | 0.34 | 0.32 | 0.34 | 0.31 | 0.29 | 0.30 |
| | | west | Corning | | | | | | | |
| | Trimethyl- | Benzoflex | Eastman | 0.78 | 0.81 | 0.83 | 0 | 1.00 | 0.97 | 0 |
| | pentanediyl | 354 (BF) | | | | | | | | |
| | Dibenzoate | | | | | | | | | |
| | Trimethyl | TXIB | Eastman | 0 | 0.50 | 0.39 | 1.36 | 0 | 0 | 0.77 |
| | Pentanyl | | | | | | | | | |
| | Diisobutyrate | | | | | | | | | |
| | Propylene | PC | | 0 | 0.55 | 0.60 | 0.18 | 0.96 | 0.97 | 0.75 |
| | Carbonate | | | | | | | | | |
| | Acrylates | Pecorez | Phoenix | 20.34 | 23.12 | 23.18 | 22.48 | 0 | 0 | 0 |
| | Copolymer And | AC50 | | | | | | | | |
| | Butyl Acetate | (50.19% | | | | | | | | |
| | | resin in | | | | | | | | |
| | | nBA) | | | | | | | | |
| | Ethyl Butyrate | EB | | 15.4 | 9.91 | 9.71 | 0 | 12.77 | 14.81 | 15.84 |
| | Butyl Acetate | nBA | | 0 | 0 | 0 | 16.63 | 0 | 6.51 | 0 |
| | Isopropyl Alcohol | IPA | | 1.86 | 0 | 0 | 0 | 0 | 0 | 0 |
| | SDA40B | | | 2.80 | 4.36 | 4.92 | 6.09 | 15.40 | 11.56 | 13.31 |
| | Iso-Butyl Acetate | iBA | | 0 | 0.59 | 0.52 | 0 | 0 | 0.63 | 0.51 |
| | Propyl Acetate | nPA | | 3.15 | 0 | 0 | 0 | 0 | 0 | 0 |
| | Methyl Butyrate | MB | | 9.37 | 19.69 | 19.58 | 0 | 25.51 | 27.69 | 23.83 |
| | Ethyl Acetate | EA | | 28.73 | 21.02 | 20.99 | 34.68 | 19.14 | 11.58 | 14.86 |
| | Methyl Propionate | MP | | 0 | 1.26 | 1.45 | 0 | 0 | 0 | 4.98 |
| B | Benzophenone-1 | B1 | Sigma | 0.10 | 0.09 | 0.10 | 0.10 | 0.10 | 0.09 | 0.10 |
| | Sucrose Benzoate | UNIPLEX | LANXESS | 2.23 | 3.96 | 3.87 | 4.03 | 5.94 | 6.16 | 6.35 |
| | | 280CG | | | | | | | | |
| C | Cellulose Acetate | CAB-381- | Eastman | 0.50 | 0.30 | 0 | 0 | 0 | 0 | 0 |
| | Butyrate | 2 | | | | | | | | |
| | Cellulose Acetate | CAB-381- | Eastman | 10.27 | 11.76 | 13.06 | 12.47 | 18.37 | 16.61 | 16.34 |
| | Butyrate | 0.5 | | | | | | | | |
| | Cellulose Acetate | CAB-551- | Eastman | 3.70 | 1.22 | 0 | 1.10 | 0 | 1.64 | 1.55 |
| | Butyrate | 0.2 | | | | | | | | |
| D | Fragrance | | | 0.48 | 0.51 | 0.49 | 0.54 | 0.50 | 0.48 | 0.52 |

| | | | | | | | |
|---|---|---|---|---|---|---|---|
| Total: | 100 | 100 | 100 | 100 | 100 | 100 | 100 |
| (density in g/cc calculated or measured | 0.998 | 1.004 | 1.002 | 0.999 | 1.008 | 1.012 | 1.007 |
| at room temperature ~21*C) S.G. | | | | | | | |
| (Calculatedfrom HSP’s) RED | 0.37 | 0.32 | 0.32 | 0.42 | 0.46 | 0.47 | 0.46 |
| (Filtered) % Solids | 33.11 | 35.72 | 35.46 | 35.77 | 30.19 | 29.90 | 29.15 |
| Tack-Free Time (min) | 1.5 | 2 | 2 | 1.5 | 1.5 | 2 | 1.5 |
| (Relative evaporation rate to reach 90% | 2.47 | 2.43 | 2.44 | 2.62 | 2.40 | 2.09 | 2.44 |
| dry film, R of nBA = 1) R 90% | | | | | | | |
| (Calculated) Flash Point (*C) | 13.5 | 14.0 | 14.0 | 13.7 | 12.7 | 15.6 | 13.1 |
| Viscosity (mPa-s, 20*C) | 913 | 984 | 977 | 889 | 969 | 897 | 762 |
| Surface Tension (Dyne/cm) | 39 | 51 | 46 | 41 | 42 | 44 | 37 |
| (Before filtering or As Received) | 3.5 | 3.0 | 2.8 | 2.9 | 3.7 | 3.6 | 3.5 |
| Transmitted % Haze (C/2) | | | | | | | |
| (After filtering) Transmitted % Haze (C/2) | 1.7 | 1.4 | 1.5 | 1.4 | 1.6 | 1.6 | 1.6 |
| 20* Gloss | 83 | 80 | 81 | 81 | 72 | 78 | 76 |
| (Reported in LogHc units, lower values | 294 | 250 | 280 | 280 | 315 | 315 | 340 |
| indicate film is less hazy) Reflected Haze | | | | | | | |
| Hardness BHN 500 g | 1.7 | 1.4 | 1.8 | 1.3 | 2.5 | 3.0 | 6.4 |
| Pencil Hardness | HB | HB | HB | HB | HB | HB | HB |
| 3 min Pull Off (mJ) | 1.7 | 1.5 | 1.7 | 1.6 | 1.2 | 1.5 | 2.2 |
| 5 min Pull Off (mJ) | 2.1 | 1.9 | 2.5 | 2.1 | 2.8 | 1.5 | 2.0 |
| (N-m) Impact/Flex | 0.14 | 0.28 | 0.14 | 0.14 | 0.28 | 0.28 | 0.42 |
| (from liquid to dry film) Shrinkage % | 66 | 63 | 64 | 64 | 73 | 74 | 72 |
| Tensile Strength (KPa) | 45 | 20 | 23 | 17 | 57 | 57 | 68 |
| Elastic Modulus (MPa) | 0.45 | 0.07 | 0.06 | 0.20 | 1.01 | 0.94 | 1.01 |

The samples in Table 9 above were made according to the procedure below:
a) Weigh out each of the part B and C ingredients into separate weighing boats and set aside.
b) Weigh out and combine the Part A ingredients in the order shown into a 200 ml HDPE pint cup.
c) Using the mixer with a 1″ saw tooth impeller, begin mixing part A at 400 RPM and add the Benzophenone-1 and Sucrose Benzoate. Mix for a period of 4 minutes until the solids are completely dissolved.
d) Next, begin slowly adding the CAB grades in the order shown while gradually increasing mixing speed from 400 RPM to 2000 RPM to maintain a vortex and prevent solids from floating and agglomerating on the surface. The CAB must be added slowly to prevent large clumps from forming which take a longer time to dissolve. It typically takes about 6 mins to add all the CAB grades and increase the mixing speed from 400 to 2000 RPM. Mix for 20 mins.
e) Stop mixing and add the fragrance (part D).
f) Decrease mix speed to 100 RPM and mix for 5 mins to blend the top coat as it de-bubbles.
g) Filter the batch according to Example 3.

All of the top coats TC-1-F through TC-7-F have better clarity (% Haze C/2), lower shrinkage %, higher pencil hardness, and lower tack-free time than Seche. Samples TC-5-F through TC-7-F have better tensile strength and indentation hardness than Seche. Samples TC-1-F, TC-4-F, TC-5-F and TC-7-F have comparable surface tension to Seche. Samples TC-1-F to TC-4-F have higher gloss and less reflective haze than Seche. Notably, sample TC-7-F has three times higher indentation hardness than Seche. All of the top coats (TC-1-F to TC-7-F) have higher indentation hardness and pencil hardness than LVX Gel Top Coat.

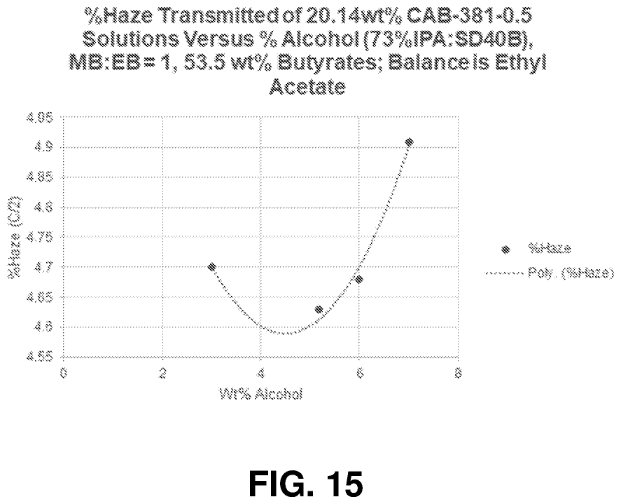

In Table 9 above, Top coat TC-4-F is an example of a top coat that does not contain butyrate esters (methyl and ethyl butyrate), yet still has excellent filtered clarity and other properties. The main ingredient choices which make this possible is maximizing the acrylates copolymer and sucrose benzoate, minimizing the CAB, and including an optimum alcohol content (see FIG. 15). Another example of a filtered top coat with good clarity which does not contain butyrate esters (SOL-1-F) is given in Table 10 below:

TABLE 10
Top coats with good filtered clarity that do not contain methyl/ethyl butyrates.

| | | | | Wt % Formula | |
|---|---|---|---|---|---|
| Part | INCI Name | Other Name | Mfg. | SOL-1-F | TC-4-F |
| A | Dimethicone | Xiameter PMX- | DOW-Corning | 0 | 0.34 |
| | | 200, 10 CSt | | | |
| | Diphenyl Dimethicone | KH54-HV | Shin-Etsu | 0.32 | 0 |
| | Trimethylpentanediyl Dibenzoate | Benzoflex 354 | Eastman | 1.22 | 0 |
| | | (BF) | | | |
| | Trimethyl Pentanyl Diisobutyrate | TXIB | Eastman | 0 | 1.36 |
| | Propylene Carbonate | PC | | 0 | 0.18 |
| | Acrylates Copolymer And Butyl | Pecorez AC50 | Phoenix | 21.17 | 22.48 |
| | Acetate | (50.19% resin in | | | |
| | | nBA) | | | |
| | Ethyl Butyrate | EB | | 0 | 0 |
| | Butyl Acetate | nBA | | 20.89 | 16.63 |
| | Isopropyl Alcohol | IPA | | 1.94 | 0 |
| | SDA40B | | | 0.70 | 6.09 |
| | Iso-Butyl Acetate | iBA | | 1.26 | 0 |
| | Propyl Acetate | nPA | | 1.91 | 0 |
| | Methyl Butyrate | MB | | 0 | 0 |
| | Ethyl Acetate | EA | | 32.90 | 34.68 |
| | Methyl Propionate | MP | | 0 | 0 |
| B | Benzophenone-1 | B1 | Sigma | 0 | 0.10 |
| | Sucrose Benzoate | UNIPLEX | LANXESS | 2.68 | 4.03 |
| | | 280CG | | | |
| C | Cellulose Acetate Butyrate | CAB-381-2 | Eastman | 0.56 | 0 |
| | Cellulose Acetate Butyrate | CAB-381-0.5 | Eastman | 10.64 | 12.47 |
| | Cellulose Acetate Butyrate | CAB-551-0.2 | Eastman | 3.81 | 1.10 |
| D | Fragrance | | | 0 | 0.54 |

| | | |
|---|---|---|
| Total: | 100 | 100 |
| (density in g/cc calculated or measured at room temperature ~21*C) S.G. | 1.007 | 0.999 |
| (Calculated from HSP's) RED | 0.45 | 0.42 |
| (Filtered) % Solids | 31.08 | 35.77 |
| Tack-Free Time (min) | 2.5 | 1.5 |
| (Calculated) Flash Point (*C) | 14.0 | 13.7 |
| Viscosity (mPa-s, 20*C) | 1032 | 889 |
| (Before filtering or As Received) Transmitted % Haze (C/2) | 3.7 | 2.9 |
| (After filtering) Transmitted % Haze (C/2) | 1.9 | 1.4 |
| 20* Gloss | 80 | 81 |
| (Reported in LogHc units, lower values indicate film is less hazy) Reflected Haze | 309 | 280 |
| Pencil Hardness | 2B | HB |
| (N-m) Impact/Flex | <0.14 | 0.14 |

The mixing procedure for SOL-1-F was to dissolve the SB for 4 mins at 400 RPM and then slowly add the CAB while increasing the speed to 2000 RPM and mix for 20 mins, followed by slow mixing at 200 RPM for 5 mins to reduce bubbles. Then, filter the batch according to Example 3.

Top coat TC-4-F is an optimized version of SOL-1, where clarity, hardness, reflected haze and impact/flex were improved upon.

### Example #6

Clear top coats TCSV66-6-1/2/3-4 were made without toluene according to the composition given in the table and procedure described below, where toluene is replaced with various acetates, alcohols, butyrates, plasticizers and other additives in various proportions. Samples TCSV66-6-1/2/3-4 include the co-resin Tosylamide/Epoxy Resin. Various chemical properties were measured and included in the Table 11 below, along with the formula.

TABLE 11
Clear topcoats with Tosylamide/Epoxy co-resin (NX55).

| | | | | Wt % Formula | | |
|---|---|---|---|---|---|---|
| | | Other | | TCSV66- | | |
| Part | INCI Name | Name | Mfg. | 6-1 | 6-2 | 6-3-4 |
| A | Trimethyl- | Benzoflex | Eastman | 0.44 | 0.41 | 0 |
| | pentanediyl | 354 (BF) | | | | |
| | Dibenzoate | | | | | |
| | Trimethyl | TXIB | Eastman | 1.13 | 1.12 | 0 |
| | Pentanyl | | | | | |
| | Diisobutyrate | | | | | |
| | Diphenyl | KF54-HV | Shin-Etsu | 0 | 0 | 0.22 |
| | Dimethicone | NX55 | Estron | 9.28 | 12.57 | 13.91 |
| | Tosylamide/ | (82.54% | | | | |
| | Epoxy Resin | resin | | | | |
| | And | in nBA) | | | | |
| | Butyl Acetate | | | | | |
| | Acrylates | Pecorez | Phoenix | 4.96 | 0 | 0 |
| | Copolymer | AC50 | | | | |
| | And Butyl | (50.19% | | | | |
| | Acetate | resin | | | | |
| | | in nBA) | | | | |
| | Ethyl | EB | | 19.25 | 22.19 | 24.23 |
| | Butyrate | | | | | |
| | Butyl Acetate | nBA | | 1.06 | 0.06 | 0.03 |
| | Isopropyl | IPA | | 1.79 | 1.79 | 1.95 |
| | Alcohol | | | | | |
| | SDA40B | | | 0.67 | 0.67 | 0.73 |
| | Iso-Butyl | iBA | | 1.18 | 1.18 | 1.26 |
| | Acetate | | | | | |
| | Methyl | MB | | 9.64 | 9.23 | 9.84 |
| | Butyrate | | | | | |
| | Ethyl Acetate | EA | | 26.17 | 26.54 | 27.21 |
| B | Benzo- | B1 | Sigma | 0.11 | 0.10 | 0.12 |
| | phenone-1 | | | | | |
| | Sucrose | UNIPLEX | LANXESS | 2.55 | 2.56 | 2.83 |
| | Benzoate | 280CG | | | | |
| | Triphenyl | TPP | | 7.04 | 7.02 | 1.45 |
| | Phosphate | | | | | |
| C | Cellulose | CAB- | Eastman | 0.52 | 0.50 | 0.55 |
| | Acetate | 381-2 | | | | |
| | Butyrate | | | | | |
| | Cellulose | CAB- | Eastman | 10.07 | 10.05 | 11.15 |
| | Acetate | 381-0.5 | | | | |
| | Butyrate | | | | | |
| | Cellulose | CAB- | Eastman | 3.59 | 3.57 | 3.95 |
| | Acetate | 551-0.2 | | | | |
| | Butyrate | | | | | |
| D | Fragrance | | | 0.52 | 0.51 | 0.57 |

| | | | |
|---|---|---|---|
| Total: | 100 | 100 | 100 |
| (density in g/cc calculated or measured | 1.020 | 1.019 | 1.010 |
| at room temperature ~21 *C) S.G. | | | |
| (Filtered) % Solids | 36.01 | 35.46 | 33.85 |
| (Calculated) Flash Point (*C) | 13.3 | 13.1 | 13.4 |
| Viscosity (mPa-s, 20*C) | 738 | 603 | 945 |
| (Before filtering) Transmitted % Haze (C/2) | 3.3 | 3.3 | 5.4 |
| (After filtering) Transmitted % Haze (C/2) | 1.4 | 1.0 | 1.3 |
| % Decrease in Transmitted % Haze (C/2) | 57 | 70 | 76 |

The samples in Table 11 above were made according to the procedure below:
a) Weigh out each of the part B and C ingredients into separate weighing boats and set aside.
b) Weigh out and combine the Part A ingredients in the order shown into a 200 ml HDPE pint cup.
c) Using the mixer with a 1″ saw tooth impeller, begin mixing part A at 400 RPM and add the Benzophenone-1 and Sucrose Benzoate. Mix for a period of 4 minutes until the solids are completely dissolved.
d) Next, add the TPP and mix for another 4 mins until the solids are completely dissolved.
e) Next, begin slowly adding the CAB grades in the order shown while gradually increasing mixing speed from 400 RPM to 2000 RPM to maintain a vortex and prevent solids from floating and agglomerating on the surface. The CAB must be added slowly to prevent large clumps from forming which take a longer time to dissolve. It typically takes about 6 mins to add all the CAB grades and increase the mixing speed from 400 to 2000 RPM. Mix for 17 mins.
f) Stop mixing and add the fragrance (part D), and mix for 3 mins at 2000 RPM.
g) Decrease mix speed to 200 RPM and mix for 5 mins to blend the top coat as it de-bubbles.
h) Filter the batch according to Example 3.

These tests seemed to show that Tosylamide/Epoxy Resin acts as a filtration aid, more so than Acrylates Copolymer, possibly due to its strong adhesive properties. The improved filtration is evidenced by a much higher decrease in % haze(C/2) than what is typically observed. By comparing top coat samples TCSV66-6-1/2, Tosylamide/Epoxy Resin also reduces haze without filtering similar to acrylates copolymer. The lot of NX-55 used in these tests has a haze % (C/2) of 1.6, compared with the lot of Pecorez AC50 used, which has a haze % (C/2) of 1.0.

### Example #7

Seeing that Tosylamide/Epoxy Resin could be used with or without Acrylates Copolymer in Example #6, a set of tests was performed to add varying wt % amounts of the co-resins to a base top coat solution (CR-1, containing only CAB resin and solvents). For each top coat sample the haze, viscosity and pencil hardness was measured before and after filtering and the % decrease in haze from filtering was calculated. The base solution CR-1 has a composition as follows in Table 12:

TABLE 12
CR-1 base top coat solution composition.

| Ingredients in CR-1 | Wt % |
|---|---|
| EA | 35.99 |
| nBA | 1.46 |
| IPA | 2.43 |
| SDA40B | 0.89 |
| MB | 12.49 |
| EB | 26.01 |
| iBA | 1.61 |
| CAB-381-0.5 | 13.6 |
| CAB-551-0.2 | 4.84 |
| CAB-381-2 | 0.68 |

The co-resins NX-55 and Pecorez AC-50 were added from 2% to 10% by weight to the CR-1 base and the composition and results of the top coat samples are given in Table 13 below:

TABLE 13
Varying the amount of co-resins NX-55 and
Pecorez AC-50. Samples with -F at the end are
the filtered top coat of that sample. (Example:
When CR2-1 is filtered it is renamed CR2-1-F).

| | NX55 | AC50 | | | | |
|---|---|---|---|---|---|---|
| | Wt % | Wt % | % | μ | Pencil | |
| | (as 100% | (as 100% | Haze | (mPa-s, | Hard- | Δ % |
| Sample# | resin) | resin) | (C/2) | 20*C) | ness | Haze |
| CRI | 0 | 0 | 5.86 | 722 | HB | — |
| CR2-1 | 2 | 0 | 7.04 | 611 | HB | — |
| CR2-2 | 6 | 0 | 5.96 | 675 | B | — |
| CR2-3 | 10 | 0 | 6.14 | 643 | B | — |
| CR3-1 | 0 | 2 | 5.71 | 627 | HB | — |
| CR3-2 | 0 | 6 | 4.53 | 627 | HB | — |
| CR3-3 | 0 | 10 | 4.21 | 691 | HB | — |
| CR1-F | 0 | 0 | 2.43 | 667 | HB | 59 |
| CR2-1-F | 2 | 0 | 2.34 | 658 | HB | 67 |
| CR2-2-F | 6 | 0 | 2.09 | 651 | HB | 65 |
| CR2-3-F | 10 | 0 | 1.83 | 635 | B | 70 |
| CR3-1-F | 0 | 2 | 2.75 | 627 | HB | 52 |
| CR3-2-F | 0 | 6 | 1.93 | 619 | HB | 57 |
| CR3-3-F | 0 | 10 | 1.61 | 707 | HB | 62 |

The above test indicates that both co-resins (Tosylamide/Epoxy Resin and Acrylates Copolymer) serve to lower the haze % in the unfiltered top coat and generally improve filtration results. The filtration results improved, generally, as the wt % addition of either co-resin was increased. The required addition level of the co-resins to lower the % haze(C/2) to below 2.1 after filtering is 6 wt %, but Acrylates Copolymer results in slightly less hazy solutions than Tosylamide/Epoxy Resin at the same addition level. Overall, the Acrylates Copolymer does work better than the Tosylamide/Epoxy Resin for lowering haze %, maintaining hardness, and achieving good film spreading and leveling. Due to the aforementioned benefits of Acrylates Copolymer, another test was conducted to measure the pencil hardness with time for top coats CR3-2-F and CR3-3-F as provided in Table 14 below:

TABLE 14
Pencil hardness with time for top coats with Acrylates Copolymer
(Dried at 22*C and 20% RH).

| Sample# | Pencil Hardness | Time (min) |
|---|---|---|
| CR-3-2-F | 2B | 20 |
| | 2B | 30 |
| | 2B | 40 |
| | HB | 60 |
| CR-3-3-F | 2B | 20 |
| | B | 30 |
| | HB | 40 |

From this Table 14 above, it appears the sample with a 10 wt % addition of Acrylates Copolymer dries somewhat faster compared to the 6 wt % addition sample and that both top coats reach the same final hardness of HB within 1 hr.

### Example #8

Based on the results of Example #7, a base solution (PR-1) was made containing solvents, CAB resins, and Acrylates Copolymer co-resin. To this base solution was added various plasticizers to a level which results in top coats that have less than 2.1% Haze(C/2) when filtered. Various chemical and mechanical properties were measured and are included in the tables below with the formulas.

TABLE 15
PR-1 base top coat solution composition.

| Ingredients in PR-1 | Wt % PR-1 |
|---|---|
| EA | 29.85 |
| nBA | 0 |
| IPA | 2.07 |
| SDA40B | 0.75 |
| MB | 10.43 |
| EB | 17.06 |
| iBA | 1.33 |
| Pecorez AC50 (50.19% resin in nBA) | 22.56 |
| CAB-381-0.5 | 11.34 |
| CAB-551-0.2 | 4.05 |
| CAB-381-2 | 0.57 |
| Total: | 100 |
| Viscosity (mPa-s, 20*C) | |
| (density in g/cc calculated or measured at room | 1.003 |
| temperature ~21*C) S.G. | |
| (Filtered) % Solids | 27.89 |
| Viscosity (mPa-s, 20*C) | 873 |
| (Before filtering) Transmitted % Haze (C/2) | 4.2 |
| (After filtering) Transmitted % Haze (C/2) | 2.0 |
| 20* Gloss | 73.2 |
| (Reported in LogHc units, lower values indicate | 322 |
| film is less hazy) Reflected Haze | |
| Pencil Hardness | HB @ 1.25 hrs |
| (N-m) Impact/Flex | <0.14 |

Top coat base solution PR-1 was made in a 700 ml HDPE cup by mixing for 20 mins at 2000 RPM while slowly adding the CAB resins and de-bubbled for 5 mins at 400 RPM.

About 50 grams of PR-1 was filtered through a 0.22 μm filter as described in previous examples above, and tested for clarity. The filtered haze % (C/2) was found to be 2.0%, which is below Seche Vite, even before any plasticizers or other additives are added to the top coat. This high clarity is in large part due to the Butyrate Esters included in the solvent and Acrylates Copolymer co-resin. Although PR-1 was made as a base solution, the testing results above show it could be used directly as a clear top coat when filtered. Various plasticizers were added to PR-1 to make the top coats with composition and chemical properties in Table 16 below:

TABLE 16
Top coats made by adding various plasticizers to base solution PR-1.

| | Wt % Filtered | | |
|---|---|---|---|
| Ingredients | PR-2-3-F | PR-3-3-F | PR-4-3-F |
| EA | 27.83 | 27.47 | 22.74 |
| IPA | 2.06 | 2.06 | 2.35 |
| SDA40B | 0.74 | 0.74 | 0.82 |
| MB | 10.18 | 10.15 | 10.64 |
| EB | 17.22 | 17.32 | 20.85 |
| iBA | 1.32 | 1.33 | 1.52 |
| TPP | 1.54 | 0 | 0 |
| TXIB | 0 | 1.52 | 0 |
| BF | 0 | 0 | 1.30 |
| Pecorez AC50 (50.19% resin in nBA) | 22.85 | 23.01 | 24.45 |
| CAB-381-0.5 | 11.55 | 11.65 | 10.89 |
| CAB-551-0.2 | 4.13 | 4.16 | 3.89 |
| CAB-381-2 | 0.58 | 0.59 | 0.55 |
| Total: | 100 | 100 | 100 |
| (density in g/cc calculated @ 20*C) S.G. | 1.003 | 1.003 | 1.003 |
| (Filtered) % Solids | 31.77 | 32 | 29.13 |
| Viscosity (mPa-s, 20*C) | 1294 | 1365 | 802 |
| (Before filtering) Transmitted | 4.2 | 4.2 | 3.6 |
| % Haze (C/2) | | | |
| (After filtering) Transmitted | 2.0 | 2.1 | 1.8 |
| % Haze (C/2) | | | |
| Pencil Hardness | HB | HB | HB |
| (N-m) Impact/Flex | <0.14 | <0.14 | <0.14 |
| Tack-Free Time (min) | 2 | 2.5 | 2 |

Top coat solutions PR-2-3-F through PR-4-3-F were created by making three incremental plasticizer additions to 75 grams of base PR-1 and taking samples in between each addition. After the third addition, the top coat solution was filtered according to Example 3 and measurements taken and results reported above.

Using a 200 ml HDPE cup, the mixing process for each addition was as follows: Mix 3 mins at 2000 RPM and de-bubble for 5 mins at 200 RPM. An exception was made for sample PR-2-3-F, which first requires 4 mins at 400 RPM to dissolve each TPP addition. Another exception was made for sample PR-4-3-F, which was adjusted to lower viscosity before filtering by adding 16 grams of solvent with composition of PR-1 solvent (namely: 6.57 g EA, 2.47 g nBA, 0.45 g IPA, 0.16 g SDA40B, 2.29 g MB, 3.76 g EB, and 0.29 g iBA)

The results for all three top coats show good hardness, tack-free time and % Haze(C/2) compared with other contemporary top coat products. The use of butyrates in the solvent enables very low levels of plasticizer to be used which results in the dried film hardness being greater than Seche Vite or LVX Gel Top Coats. It is well established that higher film hardness results in improved wear resistance and gloss over the period of one week that an air dry top coat is typically worn.

### Example #9

Based on the results of Example #8, dozens of top coats were formulated starting with base solution (PR-1) and adding various plasticizers and other additives. Various chemical and mechanical properties were measured and selected top coat results are included in table 17 below.

TABLE 17
Clear Top coats made starting from PR-1 base and adding other additives.

| | Grams Added | | | | | |
|---|---|---|---|---|---|---|
| | GL- | SR- | SR- | SR- | SR- | SR- |
| Ingredients | 3-F | 3-F | 4-F | 16-F | 22-F | 23-F |
| PR-1 Base Solution Add (g) | 75.0 | 75.0 | 75.0 | 75.05 | 75.0 | 75.0 |
| BF Add (g) | 0.95 | 0.95 | 0.95 | 0 | 0.66 | 0.66 |
| SB Add (g) | 2.15 | 2.15 | 2.15 | 0 | 1.86 | 1.86 |
| EA Add (g) | 1.0 | 1.0 | 1.0 | 0 | 1.0 | 1.0 |
| nPA Add (g) | 2.0 | 2.0 | 2.0 | 0 | 1.6 | 1.6 |
| Phenyl Trimethicone | 0 | 0.24 | 0 | 0 | 0 | 0.12 |
| (DOWSIL 556) Add (g) | | | | | | |
| Phenyl Trimethicone (CPF | 0 | 0 | 0.24 | 0 | 0 | 0 |
| 3300 10 Cst) Add (g) | | | | | | |
| Dimethicone (PMX-200 | 0 | 0 | 0 | 0 | 0.24 | 0.12 |
| 10 Cst) Add (g) | | | | | | |
| Fragrance Add (g) | 0 | 0 | 0 | 0.4 | 0.4 | 0.4 |
| Total Added (g): | 81.10 | 81.34 | 81.34 | 75.45 | 80.76 | 80.76 |
| After Mix Weight (g): | 75.26 | 78.42 | 78.90 | 73.27 | 78.1 | 78.66 |
| (Filtered) % Solids | 31.33 | 30.81 | 30.94 | 28.66 | 29.96 | 29.69 |
| Viscosity (mPa-s, 20*C) | 977 | 961 | 889 | 1048 | 881 | 889 |
| (Before filtering) Transmitted | 3.7 | 3.7 | 3.7 | 4.1 | 3.8 | 3.8 |
| % Haze (C/2) | | | | | | |
| (After filtering) Transmitted | 1.7 | 1.8 | 1.7 | 2.1 | 1.7 | 1.7 |
| % Haze (C/2) | | | | | | |
| Pencil Hardness | HB | HB | HB | HB | HB | HB |
| (N-m) Impact/Flex | <0.14 | 0.14 | <0.14 | <0.14 | 0.14 | 0.14 |
| Tack-Free Time (min) | 2 | 1.5-2 | 2-2.5 | 2 | 2 | 2 |
| 20* Gloss | 81 | 84 | 81 | 72 | 83 | 83 |
| (Reported in LogHc units) | 318 | 275 | 320 | 323 | 294 | 285 |
| Reflected Haze | | | | | | |

Using a 200 ml HDPE cup, the mixing process for each top coat in Table 17 above was as follows:
a) Add all ingredients to PR-1 Except SB.
b) Turn on mixer and dissolve SB in mixture by mixing for 4 mins at 400 RPM.
c) Mix 5 mins at 2000 RPM.
d) Debubble for 5 mins at 200 RPM.
e) Samples were filtered according to Example 3.

The results for all top coats in Table 17 above show good viscosity, clarity, hardness, tack-free time, gloss and reflective haze compared with other contemporary top coat products. The addition of dimethicone (Xiameter PMX-200 10 Cst) or Dowsil 556 (Phenyl Trimethicone) results in improved gloss, reflective haze, impact/flex resistance, and a smoother dry surface without compromising hardness, clarity or drying rate.

### Example #10

This test was designed to better understand the impact of CAB grade and butyrates on the various top coat properties being measured. Various CAB grades were dissolved in a mixture of solvents with and without butyrates and chemical properties were measured. The formulas and measurement results are shown in Table 18 below.

TABLE 18
Various CAB grades dissolved with and without butyrates.

| | Wt % Formula Makeup | | | | | | | | | |
|---|---|---|---|---|---|---|---|---|---|---|
| | CHF- | CHF- | CHF- | CHB- | CHB- | CHB- | CHB- | CHB- | CHB- | CHB- |
| Ingredients | 1 | 2 | 3 | 1 | 2 | 3 | 15 | 17 | 18 | 19 |
| EA | 38.68 | 38.71 | 40.5 | 21.38 | 21.12 | 22.12 | 21.29 | 13.37 | 0 | 22.30 |
| nBA | 38.68 | 38.72 | 40.49 | 0 | 0 | 0 | 1.01 | 8.94 | 22.32 | 0 |
| IPA | 2.40 | 2.39 | 2.51 | 4.96 | 4.90 | 5.13 | 3.66 | 3.67 | 3.65 | 3.65 |
| SDA40B | 0 | 0 | 0 | 0 | 0 | 0 | 1.38 | 1.35 | 1.35 | 1.36 |
| MB | 0 | 0 | 0 | 39.84 | 33.75 | 35.17 | 26.75 | 35.65 | 53.51 | 0 |
| EB | 0 | 0 | 0 | 13.73 | 20.17 | 21.17 | 26.76 | 17.83 | 0 | 53.52 |
| CAB-381-0.5 | 20.24 | 0 | 0 | 20.10 | 0 | 0 | 19.16 | 19.19 | 19.17 | 19.17 |
| CAB-551-0.2 | 0 | 20.18 | 0 | 0 | 20.05 | 0 | 0 | 0 | 0 | 0 |
| CAB-381-2 | 0 | 0 | 16.50 | 0 | 0 | 16.41 | 0 | 0 | 0 | 0 |
| Total: | 100 | 100 | 100 | 100 | 100 | 100 | 100 | 100 | 100 | 100 |
| Viscosity | 930 | 381 | 1207 | 786 | 397 | 1238 | 722 | 762 | 834 | 945 |
| (mPa-s, 20*C) | | | | | | | | | | |
| (Before filtering) | 4.97 | 8.05 | 7.0 | 4.63 | 8.19 | 7.56 | 4.43 | 4.52 | 4.25 | 4.81 |
| Transmitted % | | | | | | | | | | |
| Haze (C/2) | | | | | | | | | | |
| (After filtering) | 3.7 | 5.4 | 3.7 | 2.6 | 4.8 | 3.5 | — | — | — | — |
| Transmitted % | | | | | | | | | | |
| Haze (C/2) | | | | | | | | | | |
| % Decrease in | 26 | 32 | 47 | 44 | 41 | 54 | — | — | — | — |
| % Haze (C/2) | | | | | | | | | | |
| Pencil Hardness | HB | HB | HB | H | HB | B | HB | HB | HB | HB |
| (N-m) Impact/ | 0.14 | <0.14 | <0.14 | 0.14 | <0.14 | 0.14 | <0.14 | <0.14 | <0.14 | <0.14 |
| Flex | | | | | | | | | | |
| Tack-Free | 2 | 2 | 2-2.5 | 1.5 | 1.5 | 2 | 1 | 1.5 | 1.5 | 2 |
| Time (min) | | | | | | | | | | |
| 20* Gloss | 70 | 68 | 66 | 63 | 69 | 64 | 65 | 63 | 63 | 64 |
| (Reported in | 390 | 332 | 392 | 407 | 393 | 434 | 404 | 372 | 377 | 384 |
| LogHc units) | | | | | | | | | | |
| Reflected Haze | | | | | | | | | | |

The mixing process for the samples in Table 18 above was to slowly add the CAB while increasing mixing speed to 2000 RPM and mix for 20 mins, followed by mixing at 100-200 RPM for 5 mins to reduce bubbling. Top coat samples CHF-1/2/3 and CHB-1/2/3 were filtered according to Example 3.

The results in Table 18 above show that CAB-381-0.5 imparts significantly less haze to the formula than any other CAB grade. Comparing samples CHF-1 with CHB-1 shows that both the filtered and unfiltered clarity are better for the main CAB grade when butyrate esters are used in the solvent with a high ratio of MB:EB. Comparing CHF-2/3 with CHB-2/3 shows that the butyrates are not as effective at improving clarity of the unfiltered samples when the MB:EB ratio is lowered. However, comparing the filtered versions of CHF-1/2/3 and CHB-1/2/3, we see that the clarity is always better with the butyrates included regardless of the MB:EB ratio. Looking at the % decrease in % haze(C/2) for unfiltered and filtered, we can see that the butyrate ester solvents appear to act as a strong filtration aid compared with the acetate ester solvents. In comparison, it also seems possible that the MB is a better filtration aid than EB.

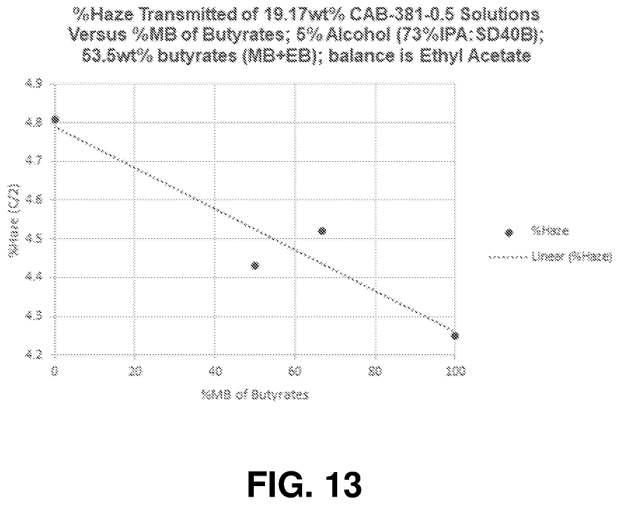

Samples CHB-15/17/18/19 further clarify the benefits of increasing the MB:EB ratio. As the wt % of MB increases and wt % of EB decreases, the % Haze(C/2) decreases for the unfiltered samples. The best clarity sample above is obtained with CHB-18, in which 100% of the butyrates are MB. This result is also presented in FIG. 13, which shows a straight line relationship between % of the total butyrate which is MB and Haze % (C/2).

### Example #11

This example is to better understand the impact of alcohol content, CAB Wt % and % MB of total butyrates on the haze % (C/2) and viscosity of a top coat containing butyrate esters in the solvent. Similar to example #10 above, CAB-381-0.5 was dissolved in a mixture of solvents with butyrates and chemical properties were measured. The formulas and measurement results are shown in Table 19 below and Table 18 above (Example #10).

TABLE 19
CAB-381-0.5 dissolved with solvents containing butyrate esters.

| | Wt % Formula Makeup | | | | | | | |
|---|---|---|---|---|---|---|---|---|
| | CHB- | CHB- | CHB- | CHB- | CHB- | CHB- | CHB- | CHB- |
| Ingredients | 8 | 9 | 10 | 11 | 12 | 13 | 14 | 16 |
| EA | 23.3 | 23.22 | 23.31 | 22.32 | 21.25 | 20.30 | 19.25 | 21.31 |
| nBA | 0 | 0 | 0 | 0 | 0 | 0 | 0 | 2.01 |
| IPA | 2.21 | 2.37 | 2.22 | 2.90 | 3.64 | 4.38 | 5.09 | 3.56 |
| SDA40B | 0.81 | 0.91 | 0.82 | 1.07 | 1.53 | 1.63 | 1.89 | 1.45 |
| MB | 35.67 | 26.69 | 17.82 | 26.77 | 26.70 | 26.77 | 26.97 | 26.75 |
| EB | 17.85 | 26.69 | 35.66 | 26.77 | 26.72 | 26.76 | 26.70 | 26.75 |
| CAB-381-0.5 | 20.17 | 20.13 | 20.18 | 20.18 | 20.16 | 20.17 | 20.11 | 18.18 |
| Total: | 100 | 100 | 100 | 100 | 100 | 100 | 100 | 100 |
| Viscosity | 1040 | 1032 | 1080 | 1017 | 1008 | 945 | 937 | 595 |
| (mPa-s, 20*C) | | | | | | | | |
| (Unfiltered) | 4.79 | 4.70 | 4.82 | 4.87 | 4.63 | 4.68 | 4.91 | 4.21 |
| Transmitted | | | | | | | | |
| % Haze (C/2) | | | | | | | | |
| Pencil Hardness | HB | HB | HB | HB | HB | HB | HB | HB |
| (N-m) Impact/Flex | <0.14 | <0.14 | 0.14 | 0.14 | <0.14 | <0.14 | <0.14 | <0.14 |
| Tack-Free | 1-1.5 | 1.5 | 1.5 | 2-2.5 | 1.5 | 1.5-2 | 1.5 | 1-1.5 |
| Time (min) | | | | | | | | |
| 20* Gloss | 64 | 65 | 67 | 64 | 66 | 66 | 66 | 63 |
| (Reported in | 403 | 403 | 394 | 404 | 383 | 386 | 401 | 425 |
| LogHc units) | | | | | | | | |
| Reflected Haze | | | | | | | | |

The mixing process for the samples in Table 19 above was to slowly add the CAB while increasing mixing speed to 2000 RPM and mix for 20 mins, followed by mixing at 100-200 RPM for 5 mins to reduce the presence of air bubbles.

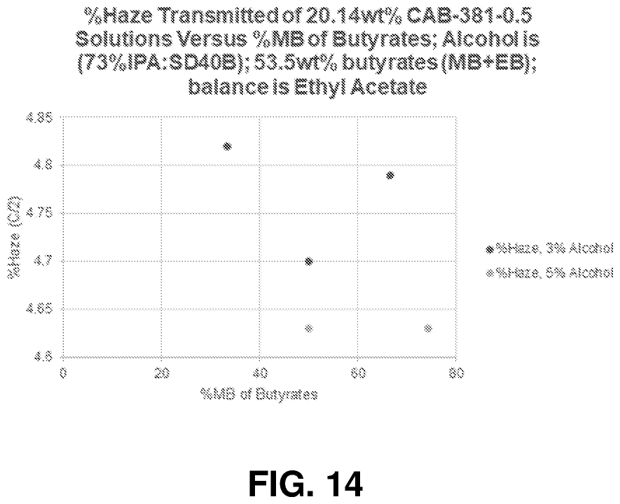

Referring to samples CHB-1/8/9/10/12 in Tables 19 (in example 11) and 18 (in example 10) above and to FIG. 14, the samples show improved clarity at 5% alcohol (where the alcohol is a mixture of IPA and SDA40B) content compared to 3% alcohol content. In comparing these samples, the CAB-381-0.5 Wt % and total butyrates Wt % were kept constant at 20.1% and 53.5%, respectively, while the % MB of total butyrates was varied.

Referring to samples CHB-9/12/13/14 in Tables 19 (above in example 11) and to FIG. 15, the data shows that there is an optimum Wt % alcohol where the Haze % (C/2) is minimized, which may change depending on other ingredients and weight percentages in any top coat formula. For this example, maximum clarity is found at about 5 wt % alcohol (the alcohol is a mixture of 73 Wt % IPA and 27 Wt % SDA40B).

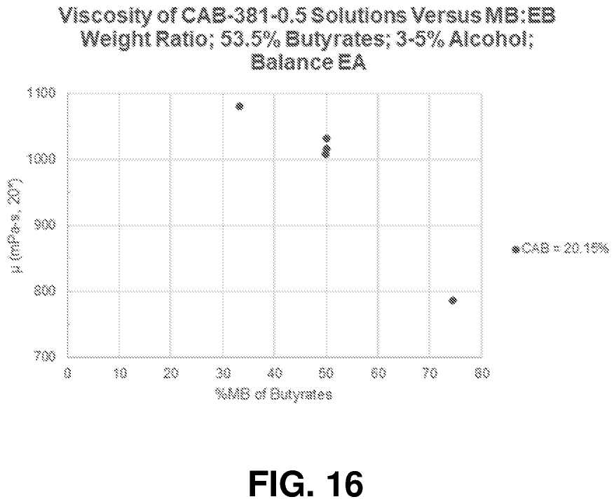

Referring to samples CHB-1/9/10/11/12 in Tables 19 (in example 11) and 18 (in example 10) above and to FIG. 16, it is observed that as the % MB of total butyrates increase the viscosity decreases, which shows MB tends to lower viscosity and EB tends to raise viscosity.

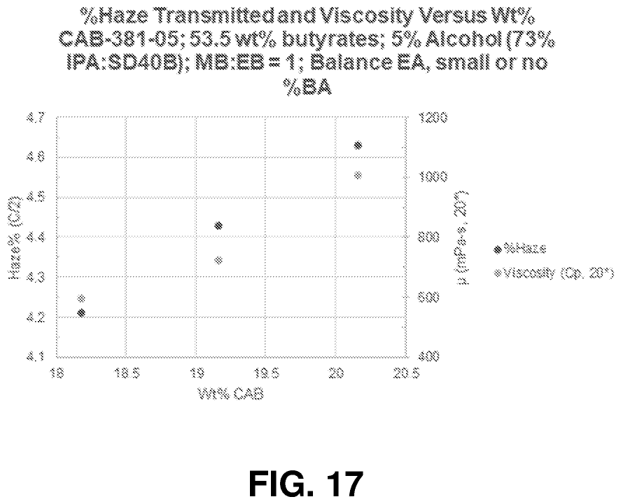

Referring to samples CHB-12/15/16 in Tables 19 (in example 11) and Table 18 (in example 10) above and to FIG. 17, it is shown that viscosity and haze increase with increasing CAB-381-0.5 Wt %. Specifically, the viscosity increases by about 400 mPa-s and the % haze (C/2) increases by 0.4 when the CAB-381-0.5 is increased by 2 Wt %.

### Example #12

This example shows it is possible to make a top coat with Haze % (C/2) equal to (Seche) 2.1 without filtering. Various grades of CAB were dissolved in a mixture of solvents (with methyl and ethyl butyrates), plasticizers and other additives, and chemical properties were measured. The formulas and measurement results are shown in Table 20 below.

TABLE 20
Clear top coat made with butyrate esters without filtering.

| | Wt % Formula Makeup | | | | |
|---|---|---|---|---|---|
| Ingredients | SR-26-F | SR-27-F | SR-28-F | SR-29-F | SR-30-F |
| EA | 0 | 0 | 0 | 7.19 | 0 |
| SDA40B | 6.88 | 4.54 | 5.42 | 4.08 | 2.24 |
| MB | 47.93 | 45.25 | 43.79 | 15.88 | 41.79 |
| EB | 0 | 0 | 0 | 16.38 | 0 |
| iBA | 0 | 0 | 0 | 1.28 | 0 |
| MP | 6.57 | 6.03 | 6.36 | 12.08 | 5.72 |
| Dimethicone, PMX-200 10 CSt | 0.31 | 0.29 | 0.31 | 0.28 | 0.29 |
| SB | 2.68 | 5.95 | 5.34 | 5.92 | 5.51 |
| PC | 0.38 | 1.08 | 1.25 | 1.06 | 1.26 |
| TXIB | 0 | 0 | 0 | 0.89 | 1.29 |
| BF | 0 | 1.94 | 1.74 | 0.96 | 1.03 |
| Fragrance | 0.49 | 0.53 | 0.50 | 0.54 | 0.55 |
| CAB-381-0.5 | 11.57 | 11.50 | 10.84 | 0 | 0 |
| CAB-551-0.2 | 0 | 0 | 0 | 3.95 | 0 |
| CAB-381-2 | 0 | 0 | 0 | 6.57 | 6.24 |
| Pecorez AC50 (50.19% Resin) | 23.20 | 22.89 | 24.45 | 22.96 | 34.08 |
| Total: | 100 | 100 | 100 | 100 | 100 |
| (Calculated) RED | 0.265 | 0.240 | 0.236 | 0.279 | 0.211 |
| Viscosity (mPa-s, 20*C) | 794 | 921 | 802 | 873 | 889 |
| (Unfiltered) Transmitted % Haze (C/2) | 2.3 | 2.3 | 2.1 | 3.8 | 2.6 |
| (Filtered) Transmitted % Haze (C/2) | 1.2 | 1.3 | 1.2 | 1.6 | 1.3 |
| Pencil Hardness | HB | HB | HB | B | 2B |
| (N-m) Impact/Flex | <0.14 | 0.42 | 0.14 | <0.14 | <0.14 |
| Tack-Free Time (min) | 2 | 2.5 | 1.5 | 2.5 | 2.5 |
| 20* Gloss | 78 | 80 | 83 | 86 | 83 |
| (Reported in LogHc units) Reflected Haze | 307 | 296 | 270 | 276 | 215 |

The mixing process for sample SR-26-F and SR-28-F in Table 20 above was to dissolve the SB for 8 mins at 400 RPM, then slowly add the CAB while increasing mixing speed to 2000 RPM and mix for 35 mins, followed by mixing at 100 RPM for 15 mins to reduce the presence of air bubbles.

The mixing process for sample SR-27-F in Table 20 above was to dissolve the SB for 4 mins at 400 RPM, then slowly add the CAB while increasing mixing speed to 2000 RPM and mix for 20 mins, followed by mixing at 100 RPM for 5 mins to reduce the presence of air bubbles.

The mixing process for sample SR-29-F in Table 20 above was to dissolve the SB for 4 mins at 400 RPM, then slowly add the CAB while increasing mixing speed to 2000 RPM and mix for 25 mins, followed by mixing at 100 RPM for 10 mins to reduce the presence of air bubbles.

The mixing process for sample SR-30-F in Table 20 above was to dissolve the SB for 4 mins at 400 RPM, then slowly add the CAB while increasing mixing speed to 2000 RPM and mix for 30 mins, followed by mixing at 100 RPM for 10 mins to reduce the presence of air bubbles.

Top coats SR-26-F through SR-30-F were filtered according to Example 3.

Sample SR-28-F in Table 20 above, has an unfiltered Haze % (C/2) of 2.1, which is equal to Seche Vite and LVX Gel top coats.

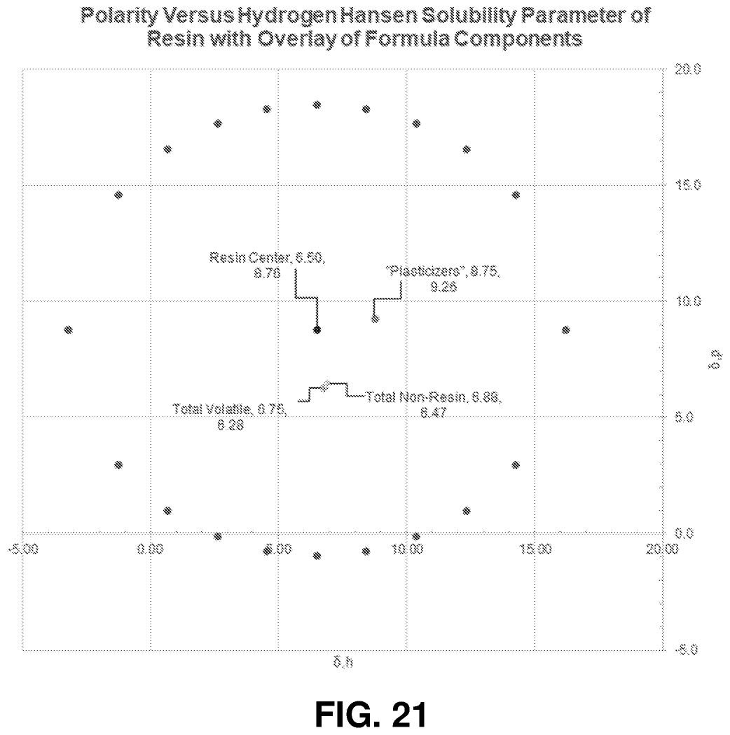

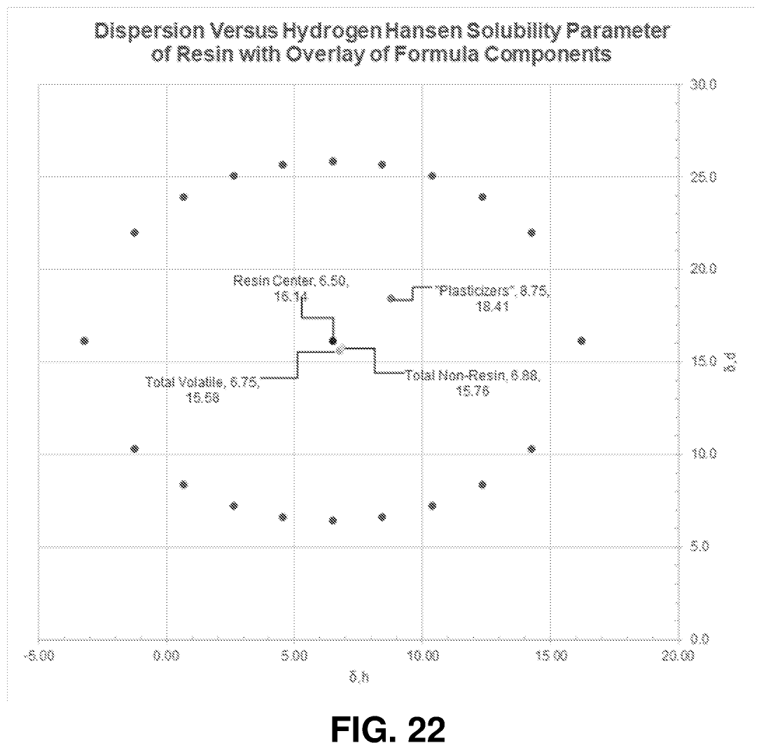

FIGS. 21 and 22 show 2D HSP plots for sample SR-28-F. The HSP plots show the calculated compatibility (dispersion, polarity and hydrogen bonding) of different parts of the top coat formula SR-28-F with the resin. The resin center consists of CAB-381-0.5 and Acrylates Copolymer, with radius Ro and coordinates shown. The “Plasticizers” includes everything in the formula that is not the resin(s) or volatile solvents, even if it is not a plasticizer: dimethicone, sucrose benzoate, propylene carbonate, benzoflex 354 and fragrance. The Total Non-Resin includes the “Plasticizers” and volatile solvents. And, the volatile solvents are n-butyl acetate, SDA40B, methyl butyrate, and methyl propionate. The dispersion parameter difference between total non-resin and resin is very small because RED is influenced four times more by dispersion than polarity or hydrogen bonding.

Referring to samples SR-29-F and SR-30-F in Table 20 above, we can see that CAB-551-0.2 and CAB-381-2 grades result in more haze and lower pencil hardness in the top coat than the other samples with only the CAB-381-0.5 grade.

Acrylates copolymer and other co-resins are useful in creating a top coat formula because they reduce the amount of CAB needed and also add clarity to the solution. The co-resins are available as a pure resin or a blend selected typically from butyl acetate, ethyl acetate and alcohol. Using a blend is ideal because it is easier to weigh and add to a formula like a thick syrup. Blending the pure co-resin with a butyrate ester is also possible as a way to add it to the top coat, which could reduce the amount of acetate esters in the top coat formula and improve clarity and perhaps other top coat properties by allowing room for more butyrate esters into the formula. Since it is common for cosmetics companies to supply resins as a blend with traditional acetate esters and alcohols (nBA, EA, IPA), it is advantageous to develop a custom blend with limited nBA and a low MB:EB ratio in order to manage odor.

According to the semi-empirical (RED/HSP) model (see FIGS. 19 and 20 and Example #15 for a description of the model), this would result in a whole range of clear top coats that doesn't need to be filtered to be at or below 2.1% haze(C/2). Below in Table 20-A are a summary of some example calculation results using the model. As shown, some or all of the nBA from SR-28-F was replaced with butyrate esters, SB and other solvents and adjustments; the haze(C/2) is predicted to be <=2.1% with a good viscosity. Utilizing the model, an endless range of formulas similar SR-28-F can be composed that don't need to be filtered, and the formulas would have about 80 to 90% likelihood of being accurate per the model's statistical data fit.

TABLE 20-A
Simulated top coats that are <=2.1% Haze (C/2) before any filtering.

| | | | Wt % Formula | | | |
|---|---|---|---|---|---|---|
| INCI | Other | | SIM- | SIM- | SIM- | SIM- |
| Name | Name | Mfg. | 1 | 2 | 3 | 4 |
| Di- | Xiameter | DOW- | 0.31 | 0.31 | 0.31 | 0.31 |
| methicone | PMX-200, | Corning | | | | |
| | 10 CSt | | | | | |
| Trimethyl- | Benzoflex | Eastman | 1.29 | 1.94 | 1.29 | 1.27 |
| pentanediyl | 354 | | | | | |
| Dibenzoate | (BF) | | | | | |
| Propylene | PC | | 1.26 | 1.25 | 1.26 | 1.24 |
| Carbonate | | | | | | |
| Acrylates | (100% | | 12.32 | 12.30 | 12.32 | 12.15 |
| Copolymer | resin) | | | | | |
| Ethyl | EB | | 29.77 | 12.92 | 20.77 | 15.55 |
| Butyrate | | | | | | |
| SDA40B | | | 2.98 | 3.80 | 2.98 | 4.32 |
| Iso-Butyl | iBA | | 1.29 | 1.29 | 1.29 | 1.27 |
| Acetate | | | | | | |
| Methyl | MB | | 20.71 | 37.47 | 29.71 | 34.23 |
| Butyrate | | | | | | |
| Butyl | nBA | | 0 | 0 | 0 | 5.11 |
| Acetate | | | | | | |
| Ethyl | EA | | 5.18 | 5.17 | 5.18 | 0 |
| Acetate | | | | | | |
| Methyl | MP | | 6.38 | 6.37 | 6.38 | 6.29 |
| Propionate | | | | | | |
| Sucrose | UNIPLEX | LANXESS | 7.12 | 5.81 | 7.12 | 7.02 |
| Benzoate | 280CG | | | | | |
| | (SB) | | | | | |
| Cellulose | CAB- | Eastman | 10.89 | 10.87 | 10.89 | 10.74 |
| Acetate | 381-0.5 | | | | | |
| Butyrate | | | | | | |
| Fragrance | | | 0.50 | 0.50 | 0.50 | 0.49 |

| | | | | |
|---|---|---|---|---|
| Total: | 100 | 100 | 100 | 100 |
| (Calculatedfrom HSP’s) RED | 0.236 | 0.218 | 0.224 | 0.226 |
| Viscosity, Calculated (mPa-s, 20*C) | 894 | 889 | 893 | 871 |
| (Before filtering, Calculated) | 2.1 | 2.1 | 2.1 | 2.1 |
| Transmitted % Haze (C/2) | | | | |

### Example #13

Various grades of CAB were dissolved in a mixture of solvents (with methyl and ethyl butyrates), plasticizers and other additives, and chemical properties were measured. The formulas and measurement results are shown in Table 21 below.

TABLE 21
Clear, Filtered top coats containing butyrate esters
made without Acrylates Copolymer.

| | Wt % Formula Makeup | | | | | | |
|---|---|---|---|---|---|---|---|
| | PCF- | PCF- | PCF- | PCF- | PCF- | PCF- | PCF- |
| Ingredients | 8-F | 12-F | 29-F | 31-F | 32-F | 33-F | 34-F |
| EA | 20.39 | 20.09 | 16.96 | 17.88 | 16.47 | 10.98 | 0 |
| nBA | 0 | 0 | 0 | 0.89 | 1.62 | 7.71 | 17.93 |
| IPA | 4.75 | 4.68 | 3.80 | 3.19 | 2.85 | 3.14 | 2.91 |
| SDA40B | 0 | 0 | 11.37 | 4.66 | 4.70 | 5.58 | 8.49 |
| MB | 38.12 | 37.56 | 22.97 | 23.05 | 21.21 | 30.12 | 42.05 |
| EB | 13.18 | 12.98 | 23.42 | 23.52 | 21.63 | 15.39 | 0 |
| iBA | 0 | 0 | 0 | 0 | 0 | 0 | 0 |
| MP | 0 | 0 | 0 | 0 | 0 | 0 | 0 |
| Diphenyl | 0.31 | 0.29 | 0 | 0 | 0 | 0 | 0 |
| Dimethicone, | | | | | | | |
| KF54-HV | | | | | | | |
| Dimethicone, | 0 | 0 | 0.28 | 0.28 | 0.26 | 0.27 | 0.25 |
| PMX-200 | | | | | | | |
| 10 CSt | | | | | | | |
| SB | 2.74 | 2.69 | 0.63 | 5.77 | 10.64 | 5.68 | 8.48 |
| PC | 0 | 0 | 1.16 | 2.89 | 4.25 | 1.17 | 2.12 |
| TXIB | 0 | 0 | 1.06 | 0 | 0 | 0 | 0 |
| BF | 1.20 | 2.67 | 0 | 0.35 | 1.06 | 1.14 | 0.74 |
| Fragrance | 0 | 0 | 0.46 | 0.46 | 0.43 | 0.45 | 0.42 |
| CAB- | 19.32 | 19.04 | 17.88 | 17.06 | 14.89 | 16.79 | 15.65 |
| 381-0.5 | | | | | | | |
| CAB- | 0 | 0 | 0 | 0 | 0 | 1.58 | 0.95 |
| 551-0.2 | | | | | | | |
| CAB-381-2 | 0 | 0 | 0 | 0 | 0 | 0 | 0 |
| Total: | 100 | 100 | 100 | 100 | 100 | 100 | 100 |
| (Calculated) | 0.553 | 0.552 | 0.510 | 0.487 | 0.443 | 0.501 | 0.465 |
| RED | | | | | | | |
| Viscosity | 945 | 969 | 620 | 754 | 548 | 915 | 722 |
| (mPa-s, | | | | | | | |
| 20*C) | | | | | | | |
| (Unfiltered) | — | 4.0 | 3.8 | 3.6 | 3.2 | 3.7 | 3.5 |
| Transmitted | | | | | | | |
| % Haze | | | | | | | |
| (C/2) | | | | | | | |
| (Filtered) | 2.1 | 2.0 | 2.0 | 1.9 | 1.8 | 1.9 | 1.8 |
| Transmitted | | | | | | | |
| % Haze | | | | | | | |
| (C/2) | | | | | | | |
| Pencil | HB | B | HB | HB | B | HB | B |
| Hardness | | | | | | | |
| (N-m) | 0.14 | <0.14 | 0.14 | 0.14 | 0.14 | 0.42 | 0.14 |
| Impact/ | | | | | | | |
| Flex | | | | | | | |
| Tack-Free | 1.5 | 1.5 | 1.5 | 2 | 2.5 | 2-2.5 | 2.5 |
| Time (min) | | | | | | | |
| 20* Gloss | 72 | 76 | 62 | 73 | 79 | 76 | 77 |
| (Reported | 408 | 399 | 374 | 324 | 248 | 339 | 278 |
| in LogHc | | | | | | | |
| units) | | | | | | | |
| Reflected | | | | | | | |
| Haze | | | | | | | |

The mixing process for all samples in Table 21 above was to dissolve the SB for 4 mins at 400 RPM, then slowly add the CAB while increasing mixing speed to 2000 RPM and mix for 10 to 15 mins until the CAB is dissolved, followed by mixing at 100 RPM for 5 mins to reduce the presence of air bubbles.

All the top coats above in Table 21 were filtered according to Example 3. The data shows slightly better filtration for the sample PCF-12-F with diphenyl dimethicone, suggesting it acts as a filtration aid.

All of the samples in Table 21 above have filtered Haze(C/2) equal or below 2.1% and do not contain acrylates copolymer. Once again, the data points to increasing clarity with decreasing CAB Wt % and increasing MB Wt %.

Referring to samples in Table 21 above, we can see that increasing SB and PC correlates with better clarity and less reflected haze, but adding too much PC lowers viscosity and hardness, while adding too much SB seems to only lower hardness. Adding too much of any plasticizer may lower hardness, as appears to be the case with BF in sample PCF-12-F. A max addition level for SB to maintain a pencil hardness of HB appears to be between 6 to 8 wt %, but may vary depending on the amount of other additives present. Similarly, a max addition level for PC to maintain a pencil hardness of HB appears to be around 2 wt %.

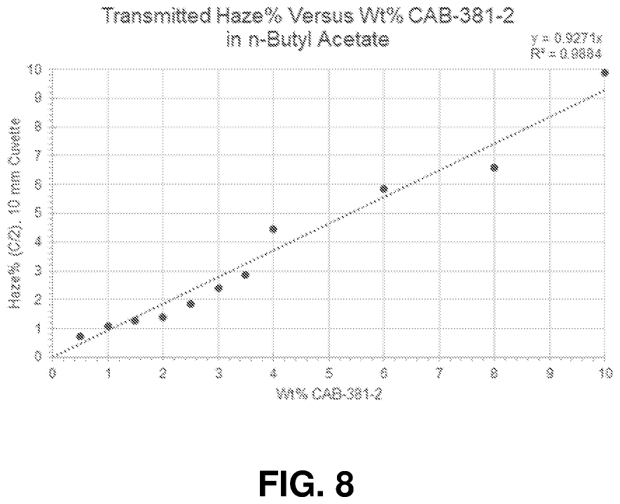

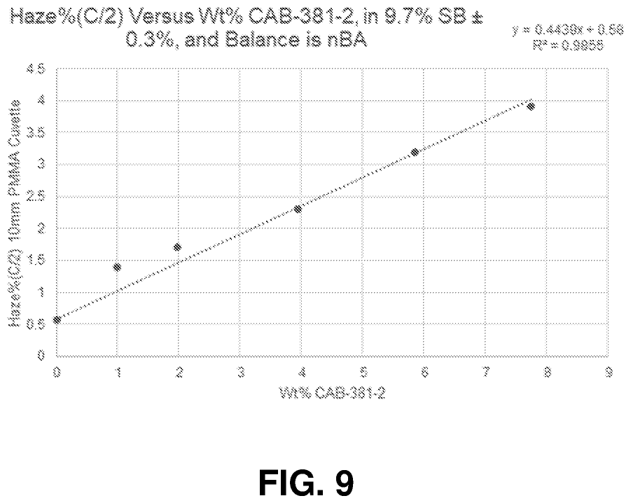

FIGS. 8 and 9 also show that incorporating Sucrose Benzoate into nBA solvent causes a sharp improvement in clarity for a given Wt % of CAB compared with the nBA solvent only.

### Example #14

This example is top coats formulated by solubility calculation according to Equations 6 and 7 above. The top coat formulas in Table 22 below were generated based on CAB having a certain solubility in each ingredient and the total CAB solubility in the top coat is additive in proportion to the product of the concentration of said ingredients and the solubility of CAB in said ingredients. The table below provides the composition and chemical properties of the top coats.

TABLE 22
Clear top coats formulated using additive solubility calculation.

| | | Wt % Formula Makeup | | | | |
|---|---|---|---|---|---|---|
| | Sᵢ (g | TCSV55- | TCSV55- | TCSV66- | TCSV66- | |
| Ingredients | CAB/g | 24-5-8 | 24-5-12 | 1-12-F | 2-8-F | C2 |
| EA | 0.03 | 0 | 0 | 20.48 | 20.01 | 21.81 |
| nBA | 0.03 | 13.11 | 16.16 | 0 | 0.38 | 0.72 |
| IPA | 0 | 0 | 0 | 0 | 0 | 0.23 |
| MB | 0.05 | 0 | 0 | 5.00 | 4.70 | 4.73 |
| EB | 0.04 | 0 | 0 | 12.96 | 12.68 | 12.91 |
| nPA | 0.06 | 43.38 | 38.59 | 0 | 0 | 0 |
| iBA | 0.04 | 0 | 0 | 1.02 | 0.99 | 1.01 |
| Methyl Acetate (MA) | 0.08 | 17.94 | 17.93 | 0 | 0 | 0 |
| Alchemix (34% NC resin: 51.86% | 0.016 | 0 | 0 | 27.80 | 27.16 | 25.75 |
| EA:IPA) ¼ Sec NC | | | | | | |
| AC50 (50.19% resin in nBA) | 0.11 | 0 | 0 | 3.62 | 3.58 | 3.37 |
| AC210 (74.74% resin in nBA) | 0.13 | 0 | 0 | 14.10 | 13.46 | 12.69 |
| SB | 0.44 | 0 | 0 | 0 | 2.00 | 1.9 |
| TPP | 0.72 | 11.93 | 9.48 | 3.50 | 3.41 | 3.24 |
| TXIB | 0.07 | 0 | 0 | 2.40 | 1.52 | 1.44 |
| BF | 0.44 | 0 | 2.62 | 2.24 | 3.41 | 3.22 |
| Lanxess Uniplex 670P . . . (78% | 0.22 | 0 | 1.97 | 0 | 0 | 0 |
| Adipic Acid/Neopentyl | | | | | | |
| Glycol/Trimellitic Anhydride | | | | | | |
| Copolymer in nBA) | | | | | | |
| *drops of 1% D&C Violet 2 | — | 0 | 0 | 0 | 0 | 0.41 |
| B1 | — | 0 | 0 | 0 | 0 | 0.1 |
| Fragrance | — | 0 | 0 | 0 | 0 | 0.50 |
| CAB-381-0.5 | — | 0 | 0 | 5.18 | 5.05 | 4.79 |
| CAB-551-0.2 | — | 0 | 0 | 1.00 | 0.48 | 0.47 |
| CAB-381-2 | — | 13.64 | 13.26 | 0.70 | 1.17 | 1.10 |

| | | | | | |
|---|---|---|---|---|---|
| Total: | 100 | 100 | 100 | 100 | 100 |
| S = Σ(SᵢXᵢ) | 0.167 | 0.161 | 0.093 | 0.106 | 0.101 |
| Wt % CAB Max = 100*[S/(1 + S)] | 14.3 | 13.9 | 8.5 | 9.6 | 9.2 |
| Total Wt % CAB | 13.64 | 13.26 | 6.88 | 6.70 | 6.36 |
| Δ = Wt % CAB Max − Total Wt % CAB | 0.67 | 0.60 | 1.62 | 2.89 | 2.83 |
| (Calculatedfrom HSP’s) RED | 0.627 | 0.62 | 0.544 | 0.535 | 0.54 |
| % Solids | 25.65 | 26.90 | 35.18 | 38.23 | 36.17 |
| Tack-Free Time (min) | 2.5 | 2.5 | 2-2.5 | 3.5 | 2.5 |
| (Calculated) Flash Point (*C) | 12.7 | 13.3 | 10.2 | 10.7 | 10.7 |
| Viscosity (mPa-s, 20*C) | 778 | 794 | 897 | 1342 | 857 |
| (Before filtering or As Received) | 3.7 | 3.2 | 4.2 | 3.5 | 2.9 |
| Transmitted %Haze (C/2) | | | | | |
| (After filtering) Transmitted % Haze (C/2) | 1.9 | 1.7 | 2 | 1.7 | 1.5 |
| 20* Gloss | 90 | 92 | 92 | 92 | 92 |
| (Reported in LogHc units, lower values | 300 | 235 | 265 | 309 | 309 |
| indicate film is less hazy) Reflected Haze | | | | | |
| Hardness BHN 500 g | 0.93 | 0.46 | 0.71 | 0.76 | 0.8 |
| Pencil Hardness | >12B | >12B | 3B | B | B |
| (N-m) Impact/Flex | <0.14 | 0.14 | 0.14 | 0.14 | 0.14 |
| (from liquid to dry film) Shrinkage % | 69 | 75 | 65 | 66 | 66 |
| Tensile Strength (KPa) | 6.4 | 4.0 | 13 | 15 | 15 |
| Elastic Modulus (MPa) | 0.02 | 0.01 | 0.1 | 0.08 | 0.08 |

The mixing process for all samples in Table 22 above was to dissolve the SB for 4 mins at 400 RPM, TPP for 3 mins at 400 RPM and then slowly add the CAB while increasing mixing speed to 2000 RPM and mix for 15 to 20 mins, followed by mixing at 100 RPM for 5 mins to reduce the presence of air bubbles. Then, filter the batch according to Example 3.

The nature of the methodology permits a wide range of possible compositions yet to be formulated. This formulation approach tends to result in too much plasticizer and not enough alcohol in the formula. The solubility calculation is not always accurate at predicting if a top coat formula is clear. The solubility of CAB in a mixture of solvents, plasticizers, and other additives may be better or worse than what the additive individual solubilities calculation predicts. Top coat “C2”, also provided in Table 8 above (Example #4), is an example of a preferred clear top coat formulated using this methodology. In the total CAB solubility (S) calculation, Xᵢ is the wt fraction of the solvent, plasticizer or other additive on a non-CAB-resin normalized basis.

The Sᵢ values in Table 22 above were determined semi-quantitatively by making solutions of individual solvents and plasticizers or other additives or binary or ternary mixtures thereof, and increasing the CAB wt % added to the mixture until a visual perception of maximum haze was reached to be considered clear. The Sᵢ value is then calculated from the known wt % of the solvent (or other additive) and the wt % of CAB, subtracting out the solubility of other components as needed for a ternary or binary mixture. The CAB grades are treated equally in this method, which is for simplification.

All of the samples in Table 22 above have Haze(C/2) equal or below 2.1% when filtered. The top coats with nitrocellulose co-resin are slightly yellow unless Violet 2 is added to neutralize the color. For top coats TCSV66-1-12-F and TCSV66-2-8-F, the excess available CAB solubility (Δ) is large, which indicates some of the plasticizers and co-resins wt % could be reduced and/or the CAB wt % increased, which may result in better hardness and other mechanical properties.

### Example #15

In a test involving filtration of dissolved CAB in a top coat, a 10 μm disk/syringe filter was used for initial filtration before using incrementally lower filter sizes to determine if greater clarity is produced at lower filter size ratings. Five samples of a cloudy formula alike to LTC-5 (Table 6) were inserted into a circular syringe filter element of various size ratings. The results are shown in FIG. 3, which provides sample names 310, filtration parameters 320, and qualitative observations 330. The sample pushed through the smallest 0.22 μm filter showed the greatest improvement of clarity and was less cloudy than all the previous filtrates. This experiment verifies that filtration is an effective method for increasing clarity of a top coat.

The above examples and supporting data represent implementations of a clear top coat that, although explicitly directed toward a cosmetics market, should be understood to potentially apply to a wide variety of substrates where it is desired to apply a clear top coat with various mechanical, optical, and chemical properties (e.g., metals, wood, shrink film, inks, electronics, personal care, and packaging).

### CITED REFERENCES

### Patent Citations

Nail Polish Top Coat Free of Toluene, U.S. Pat. No. 5,747,019, Weisman, May 5, 1998.
Nail Polish Top Coat, U.S. Pat. No. 5,130,125, Martin, et al., Jul. 14, 1992.
Non-Yellowing Rapid Drying Nail Polish Top-Coat Compositions, U.S. Pat. No. 5,785,958, Sidesai, et al., Jul. 28, 1998.
Polyethers, polyamines, polythioethers, and methods for making same, U.S. Pat. No. 10,059,801, Foley P. et al., Aug. 28, 2018.
Top Nail Coat Composition, U.S. Pat. No. 5,512,273, Martin, Apr. 30, 1996.
Universal Nail Polish Using Polyester Resin, U.S. Pat. No. 4,301,046, Schlossman, Nov. 17, 1981.
Use of nitrocellulose and cellulose ester in a nail varnish, U.S. Pat. No. 6,333,025, Ramin, Dec. 25, 2001.

### Non-Patent Citations

Heldreth, B., et al, Final Report of the Cosmetic Ingredient Review Expert Panel on the Safety Assessment of Methyl Acetate, Intl. J. of Toxicology, 21 (Supplement J) J I 2S-136S, Sage, 2012.
Becker, L. C., et al., Safety Assessment of Dimethicone Crosspolymers as Used in Cosmetics, Intl. J. of Toxicology, Vol. 33 (Supplement 2), 65S-115S, Sage, 2014.
Eastman™ Cellulose Acetate Butyrate (CAB-381-0.5) Technical Data Sheet, Eastman Chemical Company, 2018.
Eastman™ Cellulose Acetate Butyrate (CAB-381-2) Technical Data Sheet, Eastman Chemical Company, 2018.
Eastman™ Cellulose Acetate Butyrate (CAB-551-0.2) Technical Data Sheet, Eastman Chemical Company, 2018.
Eastman Solvent Selector Chart, Eastman Chemical Company, SOL-030E, 2018.
Eastman Cellulose-Based Specialty Polymers, Eastman Chemical Company, E325F, 2008.
Handling Precautions for Cellulose Esters in Formulating Coatings, Eastman Chemical Company, E-241C, 2012.
Nail Polish Formulation: Fast Drying, Clear Topcoat with Eastman Cellulose Acetate Butyrate (CAB-381-0.5) and a Polyester Resin, Eastman Chemical Company, ADD-COS-4654, 2017.
Nail Polish Formulation: Fast Drying, Clear Topcoat with Eastman CAB-381-0.5, Eastman Chemical Company, ADD-COS-020, 2015.
Hansen Solubility Parameters: A User's Handbook, 2nd Ed., Hansen, C. M., CRC Press, 2007.
Harry's Cosmeticology, Vol.'s 1-3, 9th Ed., Rosen, M. R., Editor-in-Chief, Chemical Publishing Company, 2015.
Safety Data Sheet according to the US Hazard Communication Standard (29 CFR 1910.1200), Product Name: Toluene, CAS-No: 108-88-3, EMD Millipore Corporation, Jun. 28, 2017.
Uniplex 280CG Technical Data Sheet, Product Name: sucrose benzoate, CAS-No: 12738-64-6, LANXESS Deutschland GmbH, May 16, 2012.
Bergfeld, William F., MD, et al, Final Report of the Cosmetic Ingredient Review Expert Panel on the Safety Assessment of Triphenyl Phosphate as Used in Cosmetics, Cosmetic Ingredient Review, Washington, D.C., Sep. 18, 2018.
Gry, J., Esters of Aliphatic Acyclic Primary Alcohols with Aliphatic Linear Saturated Carboxylic Acids, Safety Evaluation of Certain Food Additives and Contaminants, WHO Food Additives Series 40, The 49th Meeting of the Joint FAO/WHO Expert Committee on Food Additives (JECFA 149), World Health Organization, Geneva 1998.
Liebert et al., Cellulose Solvents: For Analysis, Shaping and Chemical Modification, ACS Symposium Series, American Chemical Society, Washington, D.C., 2010.
A. M. Api, et al., Food and Chemical Toxicology, https://doi.org/10.1016/j.fct.2019.04.058.
Bergfeld, William F., MD, et al, Final Report of the Cosmetic Ingredient Review Expert Panel on the Amended Safety Assessment of Cellulose and Related Polymers as Used in Cosmetics, Cosmetic Ingredient Review, Washington, D.C., Mar. 23, 2009.
Baines, J., et al., Evaluation of Certain Food Additives and Contaminants, 46th Report of the Joint FAO/WHO Expert Committee on Food Additives, World Health Organization, Geneva, 1997.
Standard Guide for the Comparison, Detection, and Identification of the Odors of Paints, Inks, and Related Materials, ASTM D6165-97, ASTM International, 2012.
Compilation of Odor and Taste Threshold Values Data, W. H. Stahl, Editor, ASTM Data Series DS48, ASTM, 1973.
Paint and Coating Testing Manual, J. V. Koleske, Editor, ASTM MNL 17, ASTM, 2012.
Standard Test Method for Haze and Luminous Transmittance of Transparent Plastics, ASTM D1003-13, ASTM International, 2012.
Pencil hardness ASTM D 3363-05 (2011))
Brinell ASTM E 10
https://www.totalbeauty.com/content/slideshows/non-toxic-nail-polish-170307/page18
https://www.elle.com/beauty/makeup-skin-care/news/a31283/tphp-nail-polish-study/
https://www.labmuffin.com/is-triphenyl-phosphate-tphp-in-nail-polish-dangerous/
https://www.chicagotribune.com/investigations/ct-nail-polish-toxic-chemical-20151023-story.html
https://www.sciencedirect.com/science/article/pii/S0160412015300714
https://www.ewg.org/skindeep/ingredients/706709-TRIPHENYL PHOSPHATE/

1. A clear nail top coat composition comprising:
cellulose acetate butyrate (CAB) in a proportion with one or more solvents, one or more coresin(s), and optionally plasticizers such that a top coat formed by the composition is clear.

2. The composition of claim 1, wherein one or more grades of the CAB is chosen from the following CAB grades: CAB-381-0.5, CAB-551-0.2, and CAB-381-2.

3. (canceled)

4. (canceled)

5. The composition of claim 1, wherein the one or more solvents are selected from the group consisting of: butyrates, acetates, propionates, and alcohols.

6. The composition of claim 1, wherein the one or more co-resin(s) are chosen from the group consisting of: nitrocellulose, acrylates copolymer, and tosylamide/epoxy resin.

7. The composition of claim 5, wherein the alcohols are selected from the group consisting of: ethanol, denatured alcohol (i.e. SDA40B) and IPA.

8. The composition of claim 1, wherein the plasticizers are selected from the group consisting of PC, BF, TPP, adipic acid/neopentyl glycol/trimellitic anhydride copolymer, sucrose acetate isobutyrate (SAIB), dibutyl phthalate (DBP), acetyl tributyl citrate (ATBC), and TXIB.

9. The composition of claim 1, comprising one or more smoothers selected from the group consisting of dimethicone, phenyl trimethicone, and diphenyl dimethicone.

10. The composition of claim 1, wherein the one or more solvents comprise one or more butyrate solvents.

11. The composition of claim 10, wherein the composition comprises 8 to 12% by weight of one or more grades of CAB.

12. The composition of claim 11, comprising 30 to 80% butyrate solvents.

13. The composition of claim 12, wherein the total amount of butyrate solvents comprises 30 to 100% methyl butyrate and 0 to 70% ethyl butyrate.

14. The composition of claim 12, comprising 8 to 15% acrylates copolymer by weight of the composition.

15. (canceled)

16. The composition of claim 14, comprising:
0 to 15% acetates;
1 to 7% propionates;
1 to 7% alcohol(s);
1 to 5% plasticizer(s);
4 to 8% sucrose benzoate;
0 to 0.4% smoother(s);
0 to 0.2% UV inhibitor; and
0 to 1% fragrance.

17. The composition of claim 16, wherein the composition does not require filtering to achieve a Haze(C/2) of the top coat in liquid form of <=2.1%.

18. The composition of claim 10, wherein the composition comprises 6 to 17% by weight of one or more grades of CAB and 0 to 80% butyrate solvents;
wherein the total amount of butyrate solvents comprises 0 to 100% methyl butyrate and 0 to 100% ethyl butyrate; wherein the plasticizers are selected from the group consisting of PC, BF, TPP, adipic acid/neopentyl glycol/trimellitic anhydride copolymer, sucrose acetate isobutyrate (SAIB), dibutyl phthalate (DBP), acetyl tributyl citrate (ATBC), and TXIB; wherein the composition comprises at least one of 0 to 18% acrylates copolymer, 0 to 12% tosylamide/epoxy resin, and 0 to 10% nitrocellulose; wherein the composition comprises 30 to 68% acetates, 0 to 5% propionates, 2 to 7% alcohol(s), 1 to 2% plasticizer(s), 2 to 4% sucrose benzoate,
0.0 to 0.4% smoother(s), 0.0 to 0.2% UV inhibitor, and 0.0 to 1% fragrance; and wherein the composition requires filtering to achieve a Haze(C/2) of the top coat of <=2.1%.

19. (canceled)

20. (canceled)

21. (canceled)

22. (canceled)

23. (canceled)

24. (canceled)

25. (canceled)

26. The composition of claim 1, wherein the one or more solvents are selected from the group consisting of: acetates, propionates, and alcohols; wherein the composition comprises 13 to 15% by weight of one or more grades of CAB; wherein the composition comprises at least one of 0 to 18% acrylates copolymer, 0 to 12% tosylamide/epoxy resin, and 0 to 10% nitrocellulose; wherein the composition comprises 60 to 70% acetates, 2 to 7% alcohol(s), 1-2% plasticizer(s), 2-4% sucrose benzoate, 0.0 to 0.4% smoother(s), 0.0 to 0.2% UV inhibitor, and 0.0 to 1% fragrance; and wherein the composition requires filtering to achieve a Haze(C/2) of the top coat of <=2.1%.

27. (canceled)

28. (canceled)

29. (canceled)

30. (canceled)

31. (canceled)

32. A clear nail top coat composition, comprising:
CAB in proportion with one or more solvents and optionally plasticizers such that a top coat formed by the composition is clear.

33. (canceled)

34. (canceled)

35. The composition of claim 32, wherein the one or more solvents are selected from the group consisting of: butyrates, acetates, propionates, and alcohols.

36. The composition of claim 35, wherein the alcohols are selected from the group consisting of: ethanol, denatured alcohol (i.e. SDA40B) and IPA.

37. The composition of claim 32, wherein the plasticizers are selected from the group consisting of PC, BF, TPP, adipic acid/neopentyl glycol/trimellitic anhydride copolymer, sucrose acetate isobutyrate (SAIB), dibutyl phthalate (DBP), acetyl tributyl citrate (ATBC), and TXIB.

38. The composition of claim 32, comprising one or more smoothers selected from the group consisting of dimethicone, phenyl trimethicone, and diphenyl dimethicone.

39. (canceled)

40. The composition of claim 32, wherein the composition comprises 16 to 20% by weight of one or more grades of CAB.

41. The composition of claim 32, comprising 0 to 80% butyrate solvents.

42. The composition of claim 41, wherein the total amount of butyrate solvents comprises 0 to 100% methyl butyrate and 0 to 100% ethyl butyrate.

43. (canceled)

44. The composition of claim 41, comprising:
15 to 20% acetates;
0 to 7% propionates;
11 to 17% alcohol(s);
1 to 5% plasticizer(s);
3 to 8% sucrose benzoate;
0 to 0.4% smoother(s);
0 to 0.2% UV inhibitor; and
0 to 1% fragrance.

45. The composition of claim 44, wherein the composition requires filtering to achieve Haze(C/2) <=2.1%.
